SEQUENCE LISTING (7 sequences)

SEQ ID NO: 1 - protein, 206 aa, synthetic construct
MTENRYFFAIRYLSDDVDCGLLAGRCISILHGFRQAHPGIQIGVAFPEWSDRDLGRSIAFVSTNKSLLERFRERSYFQVMQADNFFALSLVLEVPDTCQNVRFIRNQNLAKLFVGERRRRLARAKRRAKARGEAFQPHMPDETKVVGVFHSVFMQSASSGQSYILHIQKHRYERSEDSGYSSYGLASNDLYTGYVPDLGAIFSTLF

SEQ ID NO: 2 - protein, 704 aa, synthetic construct
MVTIMHIEELLDIEDHGERDRQLRRYLAPYSAEIGVDGAEKMALVVLLNLTLKRDRVESLCDEGLARQLLSDEGHITNCLHTVRWLHTHNLKYPDARVSGERLIINAPPLIPGVISSAGLPMRMGWAHDSSDINLAKLFGTSFRYRDDSTNLALQLVARSKTWEQALIGLGLTQQQLDIWCQLLASNLENNTFPTVVSPFSKQVRFLYQGNYCVVTPVVSHALLAQLQNVVHEKKLQCTYIHHDHPASVGSLVGALGGKVAVLDYPPPVSPDKARSFSQARKHRLANGQSLFDRSVFNDHVFIDALKHVISRPGLTRKQQRQLRLSALRYLRRQLAIWLGPIIEWRDEIVSSGRGEPGNLPSGGLELELITQPKKMLPELMLQVAGRFHLELQNHSAGRRFAFHPALMAPIKSQILWLLRQLADDEEKDEPHPPTSCYYLHLSGLTVYDASALANPYLCGIPSLSALAGFCHDYERRLQSLIGQSVYFRGLAWYLGRYSLVTGKHLPEPSKSADPKSVSAIRRPGLLDGRYCDLGMDLIIEVHIPTGGSLPFTTCLDLLRVALPARFAGGCLHPPSLYEEYNWCTVYQDKSTLFTVLSRLPRYGCWIYPSDADLRSFEELSEALALDRRLRPVATGFVFLEEPVERAGSIEGQHVYAESAIGTALCINPVEMRLAGKKRFFGAGFWQLNDAKGAILMNGSANTG

SEQ ID NO: 3 - protein, 410 aa, synthetic construct
MHLLVRPEPFADEALESYFLRLSQENGFERYRIFSGSVQDWLHTTDHAAAGAFPLELSRLNIFHASRSSGLRVRALQLVDRLTDGAPFRLLQLALCHSAISFGNHYKAVHRSGVDIPLSFIRVHQIPCCPDCLRESAYVRQCWHFKPYVGCHRHGGRLIYSCPACGESLNYLASESINHCQCGFDLRTASTVPAQPDEIQLSALAYGCSFESSNPLLAIGSLSARFGALYWYQQRYLSDHEAVRDDRALTKAIGHFTAWPDAFWRELQQMVDDALVRQTKPLNHTDFVDVFGSVVADCRQIPMRNTGQNFILKNLIGFLTDLVARHPQCRVANVGDLLLSAVDAATLLSTSVEQVRRLHHEGFLPLSIRPASRNTVSPHRAVFHLRHVVELRQARMQSHHDHSSTYLPAW

SEQ ID NO: 4 - protein, 347 aa, synthetic construct
MELCTHLSYSRSLSPGKAVFFYKTAESDFVPLRIEVAKISGQKCGYTEGFDANLKPKNIERYELAYSNPQTIEACYVPPNVDELYCRFSLRVEANSMRPYVCSNPDVLRVMIGLAQAYQRLGGYNELARRYSANVLRGIWLWRNQYTQGTKIEIKTSLGSTYHIPDARRLSWSGDWPELEQKQLEQLTSEMAKALSQPDIFWFADVTASLKTGFCQEIFPSQKFTERPDDHSVASRQLATVECSDGQLAACINPQKIGAALQKIDDWWANDADLPLRVHEYGANHEALTALRHPATGQDFYHLLTKAEQFVTVLESSEGGGVELPGEVHYLMAVLVKGGLFQKGKGR

SEQ ID NO: 5 - protein, 215 aa, synthetic construct
MYRRHLKHSRVKNLFKFVSAKMNTVFTVESALEFDTCFHLEYSPSVKFYEAQPEGFYYEFAGRQCPYTPDFRLVDQNDSVSFLEIKPSDKVADPDFLHRFPLKQQRAIELSSPLKLVTEKQIRIAPILGNLKLLHRYSGFQSFTPLHMQLLGLVQKLGRVSLLRLSDSIDAPPEEVLASALSLIARGIMQSDLTVQKIGISSFVWAGGHSGIDHG

SEQ ID NO: 6 - protein, 624 aa, synthetic construct
MDKHNGGLFEDEFVIPQPSTSTSPIDAIQAVLPATVDSFPYVLKVEALHRRDYILWVEKNLAGGWTEKNLTPLLADAALVLPPPTPNWRTLARWRKIYIQHGRKLVSLIPKHQAKGNARSRLPPSDELFFEQAVHRYLVGEQPSIASAFQLYSDSIRIENLGVVENPIKTISYMAFYNRIKKLPAYQVMKSRKGSYIADVEFKAIASHKPPSRIMERVEIDHTPLDLLLLDDDLLVPLGRPSLTLLIDAYSHCVVGFNLNFNQPSYESVRNALLSSISKKDYVKNKYPSIEHEWPCYGKPETLVVDNGVEFWSASLAQSCLELGINIQYNPVRKPWLKPMIERMFGIINRKLLEPIPGKTFSNIQEKGDYDPQKDAVMRFSTFLEIFHHWVIDVYHYEPDSRYRYIPIISWQHGNKDAPPAPIIGDDLTKLEVILSLSLHCTHRRGGIQRYHLRYDSDELASYRMNYPDQTRGKRKVLVKLNPRDISYVYVFLEDLGSYIRVPCIDPIGYTKGLSLQEHQINVKLHRDFINEQMDVVSLSKARIYLNDRIKNELIEVRRNIRQRNVKGVNKIAKYRNVGSHAETSIVHELNHPATNEVISKMESASQPEHCDDWDNFTSGLEPY

SEQ ID NO: 7 - protein, 335 aa, synthetic construct
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDEFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKR

(19) United States
(12) Patent Application Publication
(10) Pub. No.: US 2025/0327069 A1
(43) Pub. Date: Oct. 23, 2025

(54) ADAPTATIONS FOR HIGH EFFICIENCY AND ALTERED PAM USAGE WITH TN7-CRISPR-CAS TRANSPOSITION SYSTEMS

(71) Applicant: Cornell University, Ithaca, NY (US)

(72) Inventors: Joseph E. PETERS, Ithaca, NY (US); Elizabeth KELLOGG, Ithaca, NY (US)

(21) Appl. No.: 18/863,977

(22) PCT Filed: May 9, 2023
(86) PCT No.: PCT/US2023/066786

§ 371 Date: Nov. 7, 2024

Related U.S. Application Data
(60) Provisional application No. 63/339,807, filed on May 9, 2022.

(51) Int. Cl. C12N 15/11; C12N 9/22

(52) U.S. Cl. CPC C12N 15/111; C12N 9/224; C12N 2310/20

(57) ABSTRACT

Provided are gain-of-activity mutations in components of a type I-F3 Tn7-CRISPR-Cas system. The mutations allow use of systems that contain proteins with the mutations to function with both typical and atypical guide RNAs, and permit altered protospacer adjacent motif (PAM) usage.

### CROSS-REFERENCE TO RELATED APPLICATIONS

This application claims priority to U.S. provisional application Ser. No. 63/339,807, filed May 9, 2022, the entire disclosure of which is incorporated herein by reference.

### STATEMENT REGARDING FEDERALLY SPONSORED RESEARCH

This invention was made with government support under Grant Nos. R00-GM124463, R01GM129118, and R21AI148941, awarded by the National Institutes of Health. The Government has certain rights in the invention.

### SEQUENCE LISTING

The instant application contains a Sequence Listing, which is submitted in .xml format and is hereby incorporated by reference in its entirety. Said .xml file is named “018617_01416_ST26.xml”, was created on May 9, 2023, and is 9,844 bytes in size.

### FIELD

The present disclosure relates generally to approaches for modifying DNA, and more particularly, to improved compositions and methods for CRISPR-based editing that involve modified proteins.

### RELATED INFORMATION

CRISPR-associated transposons (CASTs) have garnered significant interest due to their capacity to direct a single cargo DNA insertion at a pre-programmed position in one orientation. These transposition systems can be highly complex: in addition to 3-4 conserved transposition genes they encode one or more CRISPR-Cas domain proteins from independent subtypes expected to carry out distinct targeting mechanisms (Peters, 2019). Early insights into the molecular adaptations that allowed CRISPR-Cas complexes to be repurposed for target site recognition came from cryo-EM structures of the I-F3a subtype, which revealed a direct physical association with a conserved transposon protein TniQ (Halpin-Healy et al., 2020). The TniQ protein works through a AAA+ regulator protein TnsC to recruit the heteromeric transposase, TnsA and TnsB (Peters, 2015). However, all target-bound complexes from CAST systems examined to date exhibit incomplete R-loop formation (Halpin-Healy et al., 2020; Li et al., 2020; Wang et al., 2020), even when reconstituted with DNA substrates designed to bypass R-loop formation (Jia et al., 2020). Therefore, important changes in the target DNA responsible for recruiting transposition components have been unclear, especially the mechanistic roles for TniQ and the extent of TniQ interactions in RNA-guided DNA transposition. Accordingly, despite the brisk activity with engineering new CRISPR-Cas genome modification tools their remains multiple unmet challenges. This is especially true with tasks that require insertion of large DNA cargos. Most strategies for integrating DNA cargo involve making a DNA double strand break with a CRISPR-Cas system and provoking the host to carry out repair using the DNA cargo with sufficient flanking homology to allow integration of the genetic information. This is an inefficient process that can also introduce unwanted ancillary mutations and additional damaging effects from inducing the host DNA damage response. There is an ongoing need for improved methods of using CRISPR systems to introduce DNA cargoes into selected locations and for making other DNA modifications. The present disclosure is pertinent to this need.

### BRIEF SUMMARY

The present disclosure provides gain-of-activity mutations in components of the type I-F3 Tn7-CRISPR-Cas system. The mutants described in this disclosure allow the same high level of transposition previously described with atypical guide RNAs, but now also with the typical guide. The disclosure also provides mutations that allow for altered PAM usage. In this regard, previous systems permitted programming of targets that are overly broad with PAM motif usage. This disclosure reveals that the I-F3 Tn7-CRISPR-Cas systems can be tuned by way of mutations in proteins of the systems to have more stringent PAM usage than other systems. The disclosure provides for improved programming of these systems by altering PAM specificity, which in certain aspects facilitates more strict PAM usage, thereby limiting off site targeting. Thus, the disclosure provides for adjusting the stringency of PAM to allow more programing options, in addition to inhibiting off-site targeting. It is expected that the representative systems and described mutations are extendable across the type I-F3 systems and may function similarly with the type I-B Tn7-CRISPR-Cas systems.

### BRIEF DESCRIPTION OF THE FIGURES

The Figures that form a part of this disclosure with this disclosure provide representative examples of constructs used for CRISPR-based engineering as described further below, and results obtained using the constructs. The disclosure includes each feature illustrated on the figures, each component of each feature individually, and all combinations thereof.

FIG. 1. High-resolution cryo-EM reveals full R-loop and partial R-loop states of the DNA-bound Cascade-TniQ complex. (A) Cryo-EM reconstructions and atomic model of the Cascade-TniQ complex in the full R-loop state (fully engaged RNA-DNA hybrid). The complex includes the following components: Cas8/5 (purple), Cas7 (green), Cas6 (olive), CRISPR RNA (crRNA, orange), TniQ (pink), and target DNA (blue). Asterisks (*) indicate the Cas8/5 helix bundle domain. Box defines the region for the close-up view in panel B. Local-resolution filtered maps from the focused refinements were combined for visualization. (B) Close-up view of the cryo-EM density and the atomic model of crRNA and target-strand DNA (tsDNA) from the full R-loop state conformation. The cryo-EM reconstruction visualizes the full engagement of tsDNA to crRNA. (C) Cryo-EM structure of the Cascade-TniQ complex in the partial R-loop state. Color scheme is identical to the panel A. Black box indicates the region for detailed view in the panel D. (D) Close-up view of PAM distal region of the R-loop from the cryo-EM structure of partial R-loop state. Base pair positions one through four are not resolved in the cryo-EM reconstruction of the partial R-loop conformation, indicated with dashed lines.

FIG. 2. PAM distal end of target DNA in full R-loop state is further unwound, interacting with TniQ and Cas8/5 helix bundle domain. (A) Atomic model of a full R-loop formed Cascade-TniQ complex, shown for reference. The color scheme is consistent with FIG. 1. TniQ is distinguished by numbers (TniQ.1 or TniQ.2) to indicate which subunit interacts with the target DNA. (B) One TniQ subunit (TniQ.1, surface representation) interacts with the PAM-distal DNA duplex (blue). DNA binding region of TniQ surface is positively charged, represented by the positive electrostatic potential. Legend indicates the color key for the dimensionless electrostatic potential calculated by APBS. (C) The PAM-distal target DNA is unwound by additional three base pairs (red) following the protospacer (dark blue). Downstream double-stranded DNA interacts with the Cas8/5 helix-bundle domain (HBD, purple) and TniQ.1 subunit. (D) Electrophoretic mobility shift assay (EMSA) reveals that TniQ promotes R-loop formation of Cascade with target DNA. At low concentrations, Cascade without TniQ shifts the DNA in a similar manner as Cascade-TniQ, but it forms a smeary band at high concentrations (>250 nM, indicated with an asterisk). On the other hand, Cascade associated with TniQ forms discrete bands in EMSA. The binding configurations that correspond to the band positions are indicated on the right of the gel image.

FIG. 3. Comparison of the crRNA bound structure of I-F3a and I-F3b point to a regulatory mechanism enacted by TniQ. (A) Schematic of spacer and target-DNA bound to Cascade-TniQ. Purple arrow indicates 5′ handle, orange indicates spacer, and star indicates 3′ stem-loop. (B) Structure of spacer and bound DNA shown in opaque surface, protein shown in transparent surface. Dotted box indicates the region shown superimposed in panel C. Symbols are as indicated in A. (C) Comparison of target-DNA bound structures of the I-F3a (6PIF, left) and I-F3b (this study, right) reveals striking differences in the structure of crRNA (blue for I-F3a, orange for I-F3b). (D) The I-F3b atomic model reveals that potential residues which may form the basis of TniQ's regulatory function. (E) Mutation of several crRNA-interacting residues abrogates the ability of I-F3b elements to regulate transposition as indicated by a restoration of typical crRNA transposition activity in a mate-out assay; data indicate mean+/−standard deviation (n=3).

FIG. 4. PAM requirements for I-F3b CAST transposition and I-F1 CRISPR-Cas interference. (A) Schematic describing the workflow of the assay. Target plasmid with 2-bp degenerate sequence upstream protospacer (orange) was sequenced before and after selection for transposition with I-F3b Tn6900 CAST or interference with I-F1 P. aeruginosa PA14 CRISPR-Cas. (B) Heatmap indicating sequence enrichment/depletion with Tn6900 transposition of sixteen possible PAM sequences. (C) Sequence enrichment/depletion with PA14 interference is shown as in B. (D-E). A subset of PAM sequences tested directly for transposition frequency in a mate-out assay (D) or interference activity by a transformation assay (E). Data indicate mean+/−standard deviation (n=3).

FIG. 5. Structure comparison and mutation analysis indicate key residues controlling PAM discrimination. (A) Comparison of Cas8 (purple) interaction with PAM bases on the target strand (blue) in the target-DNA bound structures of I-F3b (this study, left) and I-F1 (6NE0, right) indicates key differences in interacting Cas8 residues. (B) Cartoon diagram describing these interactions (I-F3b left, I-F1 right). (C) PAM wheels indicating PAM preference with I-F3b Tn6900 CAST with Cas8/5 wild-type and mutants. Preference at the −1 position is represented by the inner ring; the −2 position is represented by the outer ring. While S248A, S248N, and A247T+S248N mutants showed modest change, A247Q+S248N mutation substantially increased −1 C preference.

FIG. 6. Schematic summarizing mechanistic properties. I-F1 CRISPR surveillance complexes (purple) have stricter PAM sequence requirements, however, the I-F3 CAST family is able to make use of loosened PAM requirements. R-loop formation is accompanied by a conformational change in the Cascade complex and locks down on target-DNA via TniQ, revealing the mechanistic coupling between the CRISPR effector and core transposition protein, TniQ. This results in DNA distortions that most likely serve to recruit AAA+ regulator TnsC in order to direct DNA donor integration via the transposase, TnsA/B.

FIG. 7. Cryo-EM data processing details of Cascade-TniQ complexes. (A) Representative micrograph of DNA-bound Cascade-TniQ with atypical crRNA. Scale bar represents 100 nm. (B) Data processing workflow of Cascade-TniQ complex with partial or full R-loop. (C-D) Fourier shell correlation (FSC) curve of (C) Cascade-TniQ Full R-loop complex, and (D) Cascade-TniQ partial R-loop complex. Model-map FSC (red) and half-map FSC (blue) are estimated with and without the mask, represented in solid and dashed lines respectively. Estimated resolution based on corresponding cutoff (0.143 for half-map FSC, and 0.5 for model-map FSC) is presented. (E-G) Local resolution maps reveal variations of local resolution within the complexes. Legend on the right of each reconstruction indicates color schemes for corresponding local resolutions. (E) Local resolution map of the consensus map before 3D sorting. The cryo-EM density of TniQ and Cas8 helix bundle domain is significantly weaker, and low resolution. (F) Local resolution of Cascade-TniQ full R-loop complex reveals significantly low resolution in the TniQ region due to protein dynamics. The dashed box in the full map indicates the region for focused refinement. The estimated local resolution of the resulting cryo-EM reconstruction from focused refinement is shown at the dashed box on the right. (G) Local resolution of the Cascade-TniQ partial R-loop complex.

FIG. 8. Observed Conformational change in the Cascade-TniQ complex. The Cascade-TniQ complex extends globally during the transition from a partial R-loop conformation to a full R-loop conformation. Surface representations of full R-loop complex model (blue), and partial R-loop complex model (grey) are aligned using the Cas7 backbone. Nucleic acids and Cas8/5 helix bundle domain were removed for clarity.

FIG. 9. DNA-binding footprint of TniQ spans 18 bases. The number of bases from the end of the protospacer was counted following the footprint of TniQ, as indicated with numbers on the target-strand DNA (blue). TniQ covers 3 bases (1-3) of single-stranded DNA and 15 bases (4-18) of duplex DNA, totaling 18 bases of target DNA. Cas6 (yellow), Cas7 (green), Cas8/5 helix bundle (purple), and TniQ (pink) were represented as semi-transparent.

FIG. 10. Cas8/5 PAM-interacting residue mutations alter PAM specificity. (A) Heatmaps representing sequence enrichment/depletion for Cas8 mutants. (B). A subset of PAM sequences tested directly for transposition frequency in a mate-out assay. Data indicates mean+/−standard deviation (n=2).

FIG. 11. Rosetta simulated PAM specificity of I-F1 Cascade, I-F3b Cascade wild-type (WT), and I-F3b Cascade with Cas8/5 mutation A247Q S248N. Heatmaps representing Rosetta simulated PAM specificity values for three possible systems: (A) I-F1 Cascade (PDB 6NE0), (B) I-F3b wild-type (WT), and (C) I-F3b with A247Q S248N mutations in Cas8/5. 16 models were generated for each system using reference structures to represent all possible PAM combinations. Using the Rosetta suite, the protein-DNA interface was optimized to obtain the reported PAM specificity values. See Materials and Methods for the details of Rosetta simulation. Values in each box represent a computationally expected fraction of the Cascade bound to a specific PAM site within 16 possible PAM sites. Boxes with higher values are colored darker grey in a logarithmic scale as indicated in the legend on the right side of the heatmap.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the filing date of this application or patent.

The disclosure provides modified proteins for use in CRISPR-based DNA modification. In an example, a modified protein of the disclosure comprises a mutated Cas6 protein, a mutated TniQ protein, a mutated Cas8/5 protein, or a combination thereof. In embodiments, a modified protein of this disclosure includes a Cas6 protein comprising an amino acid change relative to a reference amino acid sequence (i.e., an endogenous or wild type sequence) at position 113 or 153, or a combination thereof. In embodiments, the change is F113A or F153A.

In one example, a modified protein of the disclosure comprises a mutated TniQ protein comprising one or more amino acid changes relative to a reference amino acid sequence at position 384, 387, 283, 330, or a combination thereof. In embodiments, the change is H384A, H387A, N283A, or R330A, or a combination thereof.

In one example, a modified protein of the disclosure comprises a mutated a Cas8/5 protein comprising one or more amino acid changes relative to a reference amino acid sequence at position 247 or 248, or a combination thereof. In embodiments, the changes comprise A247T, A247Q, S248A, S248N, or a combination thereof.

In certain embodiments, systems comprising modified proteins have different properties relative to the same system but used with unmodified proteins. Representative differences in properties relative to the wild type systems are summarized in Table A. “Atypical” and typical guide RNAs are described in PCT publication WO 2021188553 from which the entire disclosure is incorporated herein by reference. Table A includes examples of mutations that refer to amino acid sequences provided below.

TABLE A
Engineered mutant alleles in I-F3 Cascade with altered activities

| | Amino acid | |
|---|---|---|
| Protein | change | Beneficial function |
| Cas6 | F113A | Gain-of-function allele which allows high on-target |
| | | transposition without using a modified (atypical) guide RNA^{#} |
| Cas6 | F153A | Gain-of-function allele which allows high on-target |
| | | transposition without using a modified (atypical) guide RNA^{#} |
| TniQ | H384A + H387A | Gain-of-function allele set which allows high on-target |
| | | transposition without using a modified (atypical) guide RNA^{#} |
| TniQ | N283A | Gain-of-function allele which allows high on-target |
| | | transposition without using a modified (atypical) guide RNA^{#} |
| TniQ | R330A | Gain-of-function allele which allows high on-target |
| | | transposition without using a modified (atypical) guide RNA^{#} |
| Cas8/5 | S248A | Gain-of-function allele which increases PAM specificity to |
| | | reduce off-site targeting^{##} |
| Cas8/5 | S248N | Gain-of-function allele which increases PAM specificity to |
| | | reduce off-site targeting^{##} |
| Cas8/5 | A247T + S248N | Gain-of-function allele set which increases PAM specificity |
| | | to reduce off-site targeting^{##} |
| Cas8/5 | A247Q + S248N | Gain-of-function allele set which increases PAM specificity |
| | | to reduce off-site targeting and increases transposition |
| | | frequency^{##} |

*Amino acid positions are for proteins encoded by the Tn6900 element from Aeromonas salmonicida S44.

In an embodiment, Cas6 comprises or consist of the sequence with at least one of above described mutation(s):

(SEQ ID NO: 1)
MTENRYFFAIRYLSDDVDCGLLAGRCISILHGFRQAHPGIQIGVAFPEWSDRDLGRSI
AFVSTNKSLLERFRERSYFQVMQADNFFALSLVLEVPDTCQNVRFIRNQNLAKLFVG
ERRRRLARAKRRAKARGEAFQPHMPDETKVVGVFHSVFMQSASSGQSYILHIQKHR
YERSEDSGYSSYGLASNDLYTGYVPDLGAIFSTLF

In an embodiment, Cas8/5 comprises or consist of the sequence with at least one of the above described mutation(s):

(SEQ ID NO: 2)
MVTIMHIEELLDIEDHGERDRQLRRYLAPYSAEIGVDGAEKMALVVLLNLTLKRDRV
ESLCDEGLARQLLSDEGHITNCLHTVRWLHTHNLKYPDARVSGERLIINAPPLIPGVIS
SAGLPMRMGWAHDSSDINLAKLFGTSFRYRDDSTNLALQLVARSKTWEQALIGLGL
TQQQLDIWCQLLASNLENNTFPTVVSPFSKQVRFLYQGNYCVVTPVVSHALLAQLQ
NVVHEKKLQCTYIHHDHPASVGSLVGALGGKVAVLDYPPPVSPDKARSFSQARKHR
LANGQSLFDRSVENDHVFIDALKHVISRPGLTRKQQRQLRLSALRYLRRQLAIWLGPI
IEWRDEIVSSGRGEPGNLPSGGLELELITQPKKMLPELMLQVAGRFHLELQNHSAGRR
FAFHPALMAPIKSQILWLLRQLADDEEKDEPHPPTSCYYLHLSGLTVYDASALANPY
LCGIPSLSALAGFCHDYERRLQSLIGQSVYFRGLAWYLGRYSLVTGKHLPEPSKSADP
KSVSAIRRPGLLDGRYCDLGMDLIIEVHIPTGGSLPFTTCLDLLRVALPARFAGGCLHP
PSLYEEYNWCTVYQDKSTLFTVLSRLPRYGCWIYPSDADLRSFEELSEALALDRRLRP
VATGFVFLEEPVERAGSIEGQHVYAESAIGTALCINPVEMRLAGKKRFFGAGFWQLN
DAKGAILMNGSANTG

In an embodiment, TniQ comprises or consist of the sequence with at least one of the above described mutation(s):

(SEQ ID NO: 3)
MHLLVRPEPFADEALESYFLRLSQENGFERYRIFSGSVQDWLHTTDHAAAGAFPLEL
SRLNIFHASRSSGLRVRALQLVDRLTDGAPFRLLQLALCHSAISFGNHYKAVHRSGV
DIPLSFIRVHQIPCCPDCLRESAYVRQCWHFKPYVGCHRHGGRLIYSCPACGESLNYL
ASESINHCQCGFDLRTASTVPAQPDEIQLSALAYGCSFESSNPLLAIGSLSARFGALYW
YQQRYLSDHEAVRDDRALTKAIGHFTAWPDAFWRELQQMVDDALVRQTKPLNHTD
FVDVFGSVVADCRQIPMRNTGQNFILKNLIGFLTDLVARHPQCRVANVGDLLLSAVD
AATLLSTSVEQVRRLHHEGFLPLSIRPASRNTVSPHRAVFHLRHVVELRQARMQSHH
DHSSTYLPAW

In an embodiment, Cas7 comprises or consist of the sequence:

(SEQ ID NO: 4)
MELCTHLSYSRSLSPGKAVFFYKTAESDFVPLRIEVAKISGQKCGYTEGFDANLKPKN
IERYELAYSNPQTIEACYVPPNVDELYCRFSLRVEANSMRPYVCSNPDVLRVMIGLA
QAYQRLGGYNELARRYSANVLRGIWLWRNQYTQGTKIEIKTSLGSTYHIPDARRLS
WSGDWPELEQKQLEQLTSEMAKALSQPDIFWFADVTASLKTGFCQEIFPSQKFTERP
DDHSVASRQLATVECSDGQLAACINPQKIGAALQKIDDWWANDADLPLRVHEYGA
NHEALTALRHPATGQDFYHLLTKAEQFVTVLESSEGGGVELPGEVHYLMAVLVKGG
LFQKGKGR

In an embodiment, TnsA comprises or consist of the sequence:

(SEQ ID NO: 5)
MYRRHLKHSRVKNLFKFVSAKMNTVFTVESALEFDTCFHLEYSPSVKFYEAQPEGFY
YEFAGRQCPYTPDFRLVDQNDSVSFLEIKPSDKVADPDFLHRFPLKQQRAIELSSPLK
LVTEKQIRIAPILGNLKLLHRYSGFQSFTPLHMQLLGLVQKLGRVSLLRLSDSIDAPPE
EVLASALSLIARGIMQSDLTVQKIGISSFVWAGGHSGIDHG

In an embodiment, TnsB comprises or consist of the sequence:

(SEQ ID NO: 6)
MDKHNGGLFEDEFVIPQPSTSTSPIDAIQAVLPATVDSFPYVLKVEALHRRDYILWVE
KNLAGGWTEKNLTPLLADAALVLPPPTPNWRTLARWRKIYIQHGRKLVSLIPKHQA
KGNARSRLPPSDELFFEQAVHRYLVGEQPSIASAFQLYSDSIRIENLGVVENPIKTISY
MAFYNRIKKLPAYQVMKSRKGSYIADVEFKAIASHKPPSRIMERVEIDHTPLDLLLLD
DDLLVPLGRPSLTLLIDAYSHCVVGFNLNFNQPSYESVRNALLSSISKKDYVKNKYPS
IEHEWPCYGKPETLVVDNGVEFWSASLAQSCLELGINIQYNPVRKPWLKPMIERMFG
IINRKLLEPIPGKTFSNIQEKGDYDPQKDAVMRFSTFLEIFHHWVIDVYHYEPDSRYRY
IPIISWQHGNKDAPPAPIIGDDLTKLEVILSLSLHCTHRRGGIQRYHLRYDSDELASYR
MNYPDQTRGKRKVLVKLNPRDISYVYVFLEDLGSYIRVPCIDPIGYTKGLSLQEHQIN
VKLHRDFINEQMDVVSLSKARIYLNDRIKNELIEVRRNIRQRNVKGVNKIAKYRNVG
SHAETSIVHELNHPATNEVISKMESASQPEHCDDWDNFTSGLEPY

In an embodiment, TnsC comprises or consist of the sequence:

(SEQ ID NO: 7)
MDLSCHDADKLRSFIECYVETPLLRAIQEDFDRLRFNKQFAGEPQCMLLTGDTGTGK
SSLIRHYAAKHPEQVRHGFIHKPLLVSRIPSRPTLESTMVELLKDLGQFGSSDRIHKSS
AESLTEALIKCLKRCETELIIIDEFQELIENKTREKRNQIANRLKYISETAKIPIVLVGMP
WATKIAEEPQWSSRLLIRRSIPYFKLSDDRENFIRLIMGLANRMPFETQARLETKHTIY
ALFAACYGSLRALKQLLDESVKQALAAHAETLKHEHIAVAYALFYPDQVNPFLQPID
EIKACEVKQYSRYEIDAAGKEEVLNPLQFTDKIPISQLLKKR

The disclosure includes homologs and orthologs of the described sequences. In embodiments, the homolog or ortholog or a described polypeptide has a sequence identity of at least 80%, at least 85%, at least 90%, or at least 95% with a described polypeptide.

In addition to the described mutations, further modifications may comprise insertions, substitutions, or amino acids that are added to the N-terminus or C-Terminus of the described proteins. In embodiments, the mutations are relative to an endogenous sequence. By “endogenous” it is meant that a mutation comprises a replacement of a wild type amino acid sequence. In embodiments, the modification comprises a nuclear localization sequence (NLS) that functions in trafficking the modified protein to the nucleus of a cell. Suitable NLS sequence are known in the art and can be adapted for use with the proteins described herein when given the benefit of the present disclosure, One or more of the proteins may be fused together, with or without other proteins. In embodiments, Cas8 and Cas5 are present in a single fusion protein.

In embodiments, proteins described herein may be expressed from a coding sequence that includes a ribosomal skipping sequence. Ribosomal skipping sequences are known in the art and include, in non-limiting embodiments, the ribosomal skipping peptides T2A, P2A, E2A, and F2A.

It will be apparent from the accompanying figures that only some modifications of the described protein result in improved transposition, e.g., more frequent insertion of a co-delivered DNA template. In embodiments, a CRISPR system that includes one or more of the described modified proteins exhibits higher transposition frequency than a control value. The control value may be a transposition frequency obtained using one or more modified proteins that comprises a different modification than the one or more modified proteins that exhibit a higher transposition frequency, as illustrated in the accompanying figures. The modified proteins of this disclosure may also exhibit less off-target transposition than a control value. In embodiments, the described mutations permit altered PAM specificity, relative to PAM specificity exhibited by using unmodified proteins.

In embodiments, the disclosure facilitates an increase of transposition efficiency relative to a control, such as transposition from a chromosome to a plasmid, of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, fold greater than a control value. Similar transposition efficiency can be determined for transposition events where the transposition comprises transposing an element in cis, e.g., transposition from one location in a chromosome to a different location in the same chromosome.

In embodiments, the disclosure provides systems comprising the described modified proteins. The systems comprise one or more of the modified proteins, a guide RNA that is targeted to a selected location in a chromosome or plasmid, and a DNA cargo sequence.

The described systems also provides a DNA cargo sequence for use in insertion into a DNA substrate. The DNA cargo sequence can include left and right end transposon sequences. The transposon left and right end sequences may also be inserted with a DNA cargo. The DNA cargo sequence is inserted into a DNA substrate by cooperation of the described proteins and the targeting RNA to produce the DNA editing. Those skilled in the art will be able to understand the terms “left” and “right” transposon sequences, and recognize such sequences.

For use with I-F3 systems, the one or more I-F3 proteins may be obtained from, and modified, from any of organism that encode I-F3 proteins. In embodiments, an I-F3b protein that is used and/or modified according to this disclosure is encoded by the genome of an organism with an attachment site downstream of theffs gene encoding the signal recognition particle, and those that are downstream of the downstream of the rsmJgene.

In embodiments, the described modified proteins are obtained, or derived, from type any I-F3 systems, or type I-B Tn7-CRISPR-Cas systems.

The disclosure includes intact proteins described herein, and also includes functional fragments thereof. A “functional fragment” means one or more segments of contiguous amino acids of a polypeptide described herein which retain sufficient capability to participate in target RNA programmed insertion of the DNA insertion template. In embodiments, a functional fragment may therefore comprise or consist of, for example, a core domain, a catalytic domain, a polynucleotide binding domain, and the like. A single domain, or more than one domain, can be present in a functional fragment.

In embodiments, the compositions and methods of this disclosure are functional in a heterologous system. “Heterologous” as used herein means a system, e.g., a cell type, in which one or more of the components of the system are not produced without modification of the cells/system. A non-limiting embodiment of a heterologous system is any bacteria that is not Aeromonas salmonicida, including but not necessarily limited to Aeromonas salmonicida strain S44. In embodiments, a representative and non-limiting heterologous system is any type of E. coli. A heterologous system also includes any eukaryotic cell. In embodiments, the heterologous cell is a member of any group that does not endogenously use an I-F3b system.

In embodiments, the presently described systems are used to insert a DNA insertion template to virtually any position in a bacterial genome, any episomal element, or a eukaryotic chromosome, in an orientation dependent fashion, but in certain instances may require a PAM sequence. Further, the disclosure reveals by way of certain mutations and combination thereof in described proteins, the disclosure provides for altering PAM specificity.

In embodiments, the system is targeted via a targeting RNA to a sequence in a chromosome in a eukaryotic cell, or to a DNA extrachromosomal element in a eukaryotic cell, such as a DNA viral genome. Thus, the disclosure includes modifying eukaryotic chromosomes, and eukaryotic extrachromosomal elements, such as DNA in any organelle. Accordingly, the type of extrachromosomal elements that can be modified according to the presently described compositions and methods are not particularly limited.

In embodiments, systems of this disclosure include a DNA cargo for insertion into a eukaryotic chromosome or extrachromosomal element, or in the case of prokaryotes, a chromosome or a plasmid. Thus, instead of transposing an existing segment of a genome in the manner in which transposons ordinarily function, the disclosure provides for insertion of DNA cargo that can be selected by the user of the system. The DNA cargo may be provided, for example, as a circular or linear DNA molecule. The DNA cargo can be introduced into the cell prior to, concurrently, or after introducing a system of the disclosure into a cell. The sequence of the DNA cargo is not particularly limited, other than a requirement for suitable right and left ends that are recognized by proteins of the system. The right and left end sequences that are required for recognition are typically from about 90-150-bp in length. As is known in the art, such 90-150 bp length comprises multiple 22 bp binding sites for the I-F3b TnsB transposase in the element in each of the ends that can be overlapping or spaced.

The minimum length of the DNA cargo is typically about 700 bp, but it is expected that from 700 bp to 120 kb can be used and inserted. The disclosure provides for insertion of a DNA cargo without making a double-stranded break, and without disrupting the existing sequence, except for residual nucleotides at the insertion site, as is known in the art for transposons. In embodiments, the insertion of the DNA cargo occurs at a position that is from approximately 47, 48, or 49 nucleotides from a protospacer in the target (e.g., chromosome or plasmid) sequence.

Without intending to be constrained by any particular theory, it is considered that, other than a requirement for certain sequences to function with the I-F3 sequences, including but not limited to I-F3b sequences as described herein, the presently provided systems are agnostic with respect to the DNA sequence of the DNA insertion template. Accordingly, in embodiments, the DNA insertion template may be devoid of any sequence that can be transcribed, and as such may be transcriptionally inert. Such sequences may be used, for example, to alter a regulatory sequence in a genome, e.g., a promoter, enhancer, miRNA binding site, or transcription factor binding site, to result in knockout of an endogenous gene, or to provide an interval in the dsDNA substrate between two loci, and may be used for a variety of purposes, which include but are not limited to treatment of a genetic disease, enhancement of a desired phenotype, study of gene effects, chromatin modeling, enhancer analysis, DNA binding protein analysis, methylation studies, and the like.

In embodiments, the DNA sequence comprises a sequence that may be transcribed by any RNA polymerase, e.g., a eukaryotic RNA polymerase, e.g., RNA polymerase I, RNA polymerase II, or RNA polymerase III. In embodiments, the RNA that is transcribed may or may not encode a protein, or may comprise a segment that encodes a protein and a non-coding sequence that is functional. For example, functional RNAs include any catalytic RNA, or an RNA that can participate in an RNAi-mediated process. In embodiments, the functional RNA comprises all or a fragment of an siRNA, an shRNA, a tRNA, a spliceosomal RNA, or any type of micro RNA (miRNA), a snoRNA, or the like. In embodiments, the RNA that does not code for a protein encodes a long noncoding RNA (lncRNA).

In embodiments, the functional RNA may comprise a catalytic segment, and thus may be provided as a ribozyme. In embodiments, the ribozyme comprises a hammerhead ribozyme, a hairpin ribozyme, or a Hepatitis Delta Virus ribozyme. Such agents can be used, for example, to modulate any RNA to which they are targeted.

In embodiments, the DNA insertion template includes one or more promoters. The promoter may be constitutive or inducible. The promoter may be operably linked to a sequence that encodes any protein or peptide, or a functional RNA.

In embodiments, the DNA insertion template comprises one or more splice junctions. Thus, the insertion template may comprise a GU near a 5′ end of a coding sequence, and a branch site near the 3′ end of the coding sequence. In embodiments, the DNA insertion templates results in exon skipping, or it provides a mutually exclusive exon, or it provides an alternative 5′ splice junction as a donor site, or an alternative 3′ splice junction as an acceptor site, or a combination thereof. In embodiments, the DNA insertion template reduces or eliminates intron retention.

In embodiments, the DNA insertion template comprises at least one open reading frame, which may be operably linked to a promoter that is included with the DNA insertion template, or the DNA insertion template is linked to an endogenous cell promoter once integrated. The open reading frame, and thus the protein encoded by it, is not limited. In non-limiting embodiments, the DNA insertion template comprises an open reading frame that encodes a peptide, e.g., a peptide that can be translated and which may be, for example, from several to 50 amino acids in length, whereas longer sequences are considered proteins.

In embodiments, a protein encoded by the DNA insertion template includes a cellular localization signal, and thus may be transported to any particular cellular compartment. In embodiments, the encoded protein comprises a secretion signal. In embodiments, the encoded protein comprises a transmembrane domain, and thus may be trafficked to, and anchored in a cell membrane. In embodiments, the anchored protein may comprise either or both of an intracellular domain and an extracellular domain, and may accordingly be displayed on the cells surface, and may further participate in, for example, signal transduction, e.g., the protein comprises a surface receptor. In embodiments, a protein encoded by the DNA integrate template comprise a nuclear localization signal. In embodiments, a protein encoded by the DNA integrate template comprises one or more glycosylation sites.

In embodiments, the protein encoded by the DNA insertion template comprises at least one antigenic determinant, e.g., an epitope, and thus may be used to produce cells, such as antigen presenting cells, that may display a peptide comprising an epitope on the cell surface via MHC (e.g, HLA) presentation.

In embodiments, the protein encoded by the DNA insertion template encodes a binding partner, such as an antibody or antigen binding fragment of an antibody. In embodiments, the binding partner comprises an intact immunoglobulin, or as fragments of an immunoglobulin including but not necessarily limited to antigen-binding (Fab) fragments, Fab′ fragments, (Fab′)2 fragments, Fd (N-terminal part of the heavy chain) fragments, Fv fragments (two variable domains), dAb fragments, single domain fragments or single monomeric variable antibody domains, isolated CDR regions, single-chain variable fragment (scFv), and other antibody fragments that retain antigen binding function. In embodiments, one or more binding partners are encoded by the DNA insertion template and encode all or a component of a Bi-specific T-cell engager (BiTE), a bispecific killer cell engager (BiKE), or a chimeric antigen receptor (CAR), such as for producing chimeric antigen receptor T cells (e.g. CAR T cells). In embodiments, the binding partners are multivalent, and as such may include tri-specific antibodies or other tri-specific binding partners.

In embodiments, the DNA insertion template encodes a T cell receptor, and thus may encode both an alpha and beta chain T cell receptor, or separate DNA insertion template s may be used.

In embodiments, the DNA insertion template encodes an enzyme; a structural protein; a signaling protein, a regulatory protein; a transport protein; a sensory protein; a motor protein; a defense protein; or a storage protein. In embodiments, the DNA insertion template encodes a protein or peptide hormone. In embodiments, the DNA insertion template encodes hemoglobin. In embodiments, the DNA insertion template encodes all or a segment of dystrophin. In embodiments, the DNA insertion template encodes a rod or cone protein. In embodiments, the DNA insertion template encodes a selectable or detectable marker. In embodiments, the detectable marker comprises a fluorescent protein, such as green fluorescent protein (GFP), enhanced GFP (eGFP), mCherry, and the like. In embodiments, the DNA insertion template encodes an auxotrophic marker, such as for use in yeast. In embodiments, the DNA insertion template encodes one or more proteins that are involved in a metabolic pathway.

In embodiments, the DNA insertion template encodes a peptide or protein that is intended to stimulate an immune response, which may be a humoral and/or cell mediated immune response, and may also include a peptide or protein that is intended to induce tolerance, such as in the case of an autoimmune disease or an allergy. In embodiments, the DNA insertion template encodes a Toll-like-receptor (TLR), or a TLR ligand, which may be an agonist or an antagonistic TLR ligand.

In embodiments, the DNA insertion template comprises a sequence that is intended to disrupt or replace a gene or a segment of a gene. Thus, the disclosure includes producing both knock in and knock out gene modifications in cells, and transgenic non-human animals that contain such cells, as well as prokaryotic cells modified in a similar manner.

In embodiments, the transposable DNA cargo sequence is inserted into the chromosome or extrachromosomal element within a 5 nucleotide sequence that includes the nucleotide that is located 47 nucleotides 3′ relative to the 3′ end of the protospacer. In embodiments, a DNA cargo insertion comprises an insertion at the center of a 5 bp target site duplication (TSD). Thus, in non-limiting embodiments, a suitable guide RNA directs an editing complex to a DNA target comprising PAM that is cognate to the protospacer, so that precise integration of a DNA cargo can be achieved. In embodiments, the PAM comprises or consists of TACC or CC, NC, or CN (where “N” is any nucleotide except A).

The I-F3b transposon and I-F3b Cas genes, or those from any other suitable system, can be expressed from any of a wide variety of existing mechanism that can replicate separately in the cell or be integrated into the host cell genome. Alternatively, they could be expressed transiently from an expression system that will not be maintained. In certain embodiments, the proteins themselves could be directly transformed into the host strain to allow their function. The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of I-F3b genes tnsA, tnsB, tnsC, and one or more I-F3b tniQ genes, and I-F3b Cas genes cas8f cas5f cas7f and cas6f, with at least one of the described mutations, and a sequence encoding at least a first guide RNA that is functional with I-F3b proteins encoded by the Cas genes, wherein at least one of the first set of I-F3b transposon genes, the I-F3b Cas genes, or the sequence encoding the first guide RNA are present within and/or are encoded by a recombinant polynucleotide that is introduced into heterologous bacteria, or eukaryotic cells. The disclosure thus includes second, third, fourth, fifth, or more copies of distinct I-F3b transposon genes, I-F3b Cas genes, and distinct cargo coding sequences.

The delivery vector can be based on any number of plasmid, bacteriophage or another genetic element, when used in prokaryotes. The vector can be engineered so it is maintained, or not maintained (using any number of existing plasmid, bacteriophage or other genetic elements). Delivery of these DNA constructions in bacteria can be by conjugation, bacteriophage or any transformation processes that functions in the bacterial host of interest.

Modifications of this system may include adapting the expression system to allow expression in eukaryotic or archaeal hosts. In embodiments, for eukaryotic cells, the disclosure includes use of at least one NLS in one or more proteins.

In embodiments, a system of this disclosure is introduced into eukaryotic cells using, for example, one or more expression vectors, or by direct introduction of ribonucleoproteins (RNPs). In embodiments, expression vectors comprise viral vectors. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as a lentiviral vector. In embodiments, a baculovirus vector may be used. In embodiments, any type of a recombinant adeno-associated virus (rAAV) vector may be used. In embodiments, a recombinant adeno-associated virus (rAAV) vector may be used. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure.

Further modification of this approach can include expression and isolation of the proteins required for this process and carrying out some or all of the process in vitro to allow the assembly of novel DNA substrates. These DNA substrates can subsequently be delivered into living host cells or used directly for other procedures. Thus, the disclosure includes compositions, methods, vectors, and kits for use in the present approach to DNA editing.

In one example, the disclosure provides a system for modifying a genetic target in bacteria and/or eukaryotic cells. The system comprises a first set of I-F3b transposon genes tnsA, tnsB, tnsC, one or more I-F3b tniQ, Cas genes cas8f cas5f cas7f, and cas6f wherein at least one of the proteins is modified as described herein, and a sequence encoding a guide RNA as described herein that is functional at least with proteins encoded by the I-F3b Cas genes, wherein at least one of the first set of transposon genes, the Cas genes, and/or or the sequence encoding the first guide RNA are present within and/or are encoded by a recombinant polynucleotide.

In embodiments, use of the described I-F3b systems exhibit a greater transposition frequency than transposition reference frequency. In embodiments, for instance in bacteria, transposition frequency can be determined using, for example, a bacteriophage (i.e. viral) vector that cannot replicate or integrate into the bacterial strain used in the assay. Therefore, while the viral vector injects its DNA into the cell, it is lost during cell replication. Encoded in the phage DNA is a miniature Tn7 element where the Right and Left ends of the element flank a gene that encodes resistance to an antibiotic, such as Kanamycin (KanR). If the transposon remains on the bacteriophage DNA the cell will still be killed by the antibiotic because the bacteriophage cannot be maintained in that particular strain of bacteria. However if the TnsA, TnsB, TnsC and other required I-F3b transposon proteins and nucleotide sequences described herein are added to the cell, transposition will occur because the transposon can move from the bacteriophage DNA into the chromosome (or plasmid) where it will be maintained and allow a colony of bacteria to grow that is antibiotic resistant. Therefore, when the number of infectious bacteriophage particles are in the assay is known, it permits calculation of a frequency of transposition as antibiotic resistant colonies of bacteria per bacteriophage used in the experiment. Thus, in embodiments, using one or a combination of the I-F3b proteins described herein increases transposition frequency. Accordingly, in some embodiments, one or more I-F3b proteins and guide RNA elements as described herein may be used to enhance CRISPR mediated insertion that is accompanied by the transposon-based constructs that are described herein.

In alternative embodiments, detectable markers and selection elements can be used. In embodiments, transposition frequency can be measured, for example, by a change in expression in a reporter gene. Any suitable reporter gene can be used, non-limiting examples of which include adaptations of standard enzymatic reactions which produce visually detectable readouts. In embodiments, adaptations of P-galactosidase (LacZ) assays are used. In embodiments, transposition of an element from one chromosomal location to another, or from a plasmid to a chromosome, or from a chromosome to a plasmid, results in a change in expression of a reporter protein, such as LacZ. In embodiments, use of a system described herein causes a change in expression of LacZ, or any other suitable marker, in a population of cells. In embodiments, transposition efficiency is determined by measuring the number of cells within a population that experience a transposition event, as determined using any suitable approach, such as by reporter expression, and/or by any other suitable marker and/or selection criteria. In embodiments, the disclosure provides for increased transposition, such as within a population of cells, relative to a control. As described above, the control can be any suitable control, such as a reference value, or any value using a control experiment with proteins that have different modifications. In embodiments, the reference value comprises a standardized curve(s), a cutoff or threshold value, and the like. In embodiments, transposition efficiency comprises use of a system of this disclosure to transpose all or a segment of DNA from one location to another within the same or separate chromosomes, from a chromosome to a plasmid, or from a plasmid or other DNA cargo to a chromosome. In embodiments, transposition efficiency is greater than a control value obtained or derived from transposition efficiency using the described system.

In one aspect, the disclosure provides a system for modifying a genetic target in one or more cells, the system comprising a first set of transposon genes tnsA, tnsB, tnsC, and tniQ, Cas genes cas8f, cas5f cas7f and cas6f which encode at least one modified protein as described herein, and wherein at least two of said proteins are within a fusion protein, and a sequence encoding a guide RNA polynucleotide.

In another embodiment the disclosure provides a method comprising expressing a guide RNA in cells comprising transposon genes tnsA, tnsB, tnsC, wherein the encoded TnsC protein comprises a modification, and wherein and optionally the TnsA and TnsB proteins are present in a described fusion protein, non-limiting examples of which are provided by the Figures.

In certain approaches of this disclosure expression vectors, such as plasmids, are used to produce one or more than one construct and/or component of the system, and any of their cloning steps or intermediates. A variety of suitable expression vectors known in the art can be adapted to produce components of this disclosure, including vectors that contain any desirable cargo, but in the context of other components described herein, and atypical repeats.

In embodiments, any protein of this disclosure may be an Aeromonas salmonicida strain S44 protein, or a derivative thereof,

The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of transposon genes tnsA, tnsB, tnsC, and optionally one or more tniQ genes, Cas genes cas8f cas5f cas7f and cas6f, encoding the described proteins with at least one, or any combination of described mutations, and a sequence encoding a guide RNA that is functional with proteins encoded by the Cas genes, wherein at least one of the first set of transposon genes, the Cas genes, or the sequence encoding the first guide RNA are present within and/or are encoded by a recombinant polynucleotide that is introduced into bacteria, or eukaryotic cells. The disclosure thus includes second, third, fourth, fifth, or more copies of distinct transposon genes, Cas genes, and distinct cargo coding sequences.

In one example, the disclosure provides a system for modifying a genetic target in bacteria and/or eukaryotic cells. The system comprises a first set of transposon genes tnsA, tnsB, tnsC, and optionally one or more tniQ, Cas genes cas8f cas5f cas7f and cas6f, encoding the described proteins with at least one, or any combination of described mutations, and a sequence encoding a first guide RNA, as described herein, that is functional with proteins encoded by the Cas genes, wherein at least one of the first set of transposon genes, the Cas genes, and/or or the sequence encoding the a guide RNA are present within and/or are encoded by a recombinant polynucleotide.

In embodiments, certain proteins that are provided by this disclosure comprise mutations relative to a wild type sequence. A “wild type” sequence as used herein means a sequence that preexists in nature without experimentally engineering a change in the sequence. In embodiments, a wild type sequence is the sequence of a transposition element, a non-limiting example of which is the sequence ofAeromonas salmonicida strain S44 plasmid pS44-1, which can be accessed via accession no. CP022176 (Version CP022176.1), such as via www.ncbi.nlm.nih.gov/nuccore/CP022176.

Non-limiting embodiments of amino acid sequences comprising mutations and/or locations of mutations are described herein, and by way of the following amino acid sequences and accession numbers.

In one aspect the disclosure includes a kit comprising one or more expression vector(s) that encodes one or more Cas or other enzymes described herein. The expression vector in certain approaches includes a cloning site, such as a poly-cloning site, such that any desirable cargo gene(s) can be cloned into the cloning site to be expressed in any target cell into which the system is introduced or already comprises. The kit can further comprise one or more containers, printed material providing instructions as to how to use make and/or use the expression vector to produce suitable vectors, and reagents for introducing the expression vector into cells. The kits may further comprise one or more bacterial strains for use in producing the components of the system. The bacterial strains may be provided in a composition wherein growth of the bacteria is restricted, such as a frozen culture with one or more cryoprotectants, such as glycerol. In embodiments, the kit comprises a vector for expression of a guide RNA comprising a user selected spacer.

In another aspect the disclosure comprises delivering to cells a DNA cargo via a system of this disclosure. The method generally comprises introducing one or more polynucleotides of this disclosure, or a mixture or proteins and polynucleotides encoding the proteins, which may be also provided with RNA polynucleotides, such as the presently described guide RNAs, into one or more bacterial or eukaryotic cells, whereby the Cas and transposon enzymes/proteins are expressed and editing of the chromosome or another DNA target by a combination of the Cas enzymes and the transposon occurs.

In non-limiting embodiments, this disclosure is considered to be suitable for targeting eukaryotic cells, and any microorganism that is susceptible to editing by a system as described herein. In embodiments the microorganism comprises bacteria that are resistant to one or more antibiotics, whereby the editing by the present system kills or reduces the growth of the antibiotic-resistant bacteria, and/or the system sensitizes the bacteria to an antibiotic by, for example, use of cargo that targets an antibiotic resistance gene, which may be present on a chromosome or a plasmid. The disclosure is thus suitable for targeting bacterial chromosomes or episomal elements, e.g., plasmids. In embodiments, a modification of a bacterial chromosome or plasmid causes the bacteria to change from pathogenic to non-pathogenic.

In embodiments, bacteria are killed. In embodiments, one or all of the components of a system described herein can be provided in a pharmaceutical formulation. Thus, in embodiments, DNA, RNA, proteins, and combinations thereof can be provided in a composition that comprises at least one pharmaceutically acceptable additive.

In embodiments, the method of this disclosure is used to reduce or eradicate bacterial cells, and may be used to reduce or eradicate persister bacteria and/or dormant viable but non-culturable (VBNC) bacteria from an individual or an inanimate surface, or a food substance.

In embodiments, and as noted above, the disclosure is considered suitable for editing eukaryotic cells. In embodiments, eukaryotic cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made. In embodiments, the cells are mammalian cells. In embodiments, the cells are human, or are non-human animal cells. In embodiments, the non-human eukaryotic cells comprise fungal, plant or insect cells. In one approach the cells are engineered to express a detectable or selectable marker, or a combination thereof.

In embodiments, the disclosure includes obtaining cells from an individual, modifying the cells ex vivo using a CRISPR system as described herein, and reintroducing the cells or their progeny into the individual for prophylaxis and/or therapy of a condition, disease or disorder, or to treat an injury, trauma or anatomical defect. In embodiments, the cells modified ex vivo as described herein are used autologously.

In embodiments, cells modified according to this disclosure are provided as cell lines. In embodiments, the cells are engineered to produce a protein or other compound, and the cells themselves or the protein or compound they produce is used for prophylactic or therapeutic applications.

In various embodiments, the modification introduced into eukaryotic cells according to this disclosure is homozygous or heterozygous. In embodiments, the modification comprises a homozygous dominant or homozygous recessive or heterozygous dominant or heterozygous recessive mutation correlated with a phenotype or condition, and is thus useful for modeling such phenotype or condition. In embodiments a modification causes a malignant cell to revert to a non-malignant phenotype.

In certain aspects the disclosure includes a pharmaceutical formulation comprising one or more components of a system described herein. A pharmaceutical formulation comprises one or more pharmaceutically acceptable additives, many of which are known in the art. In some embodiments, the pharmaceutical compositions comprise a pharmaceutically acceptable carrier suitable for administration to humans. In some embodiments, the pharmaceutical compositions comprise a pharmaceutically acceptable carrier suitable for intraocular injection. In some embodiments, the pharmaceutical compositions comprise a pharmaceutically acceptable carrier suitable for topical application. In some embodiments, the pharmaceutical compositions comprise a pharmaceutically acceptable carrier suitable for intravenous injection. In some embodiments, the pharmaceutical compositions comprise and a pharmaceutically acceptable carrier suitable for injection into arteries. In some embodiments, the pharmaceutical composition is suitable for oral or topical administration. All of the described routes of administration are encompassed by the disclosure.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference. Further, any of a variety of therapeutic delivery agents can be used, and include but are not limited to nanoparticles, lipid nanoparticle (LNP), exosomes, and the like. In embodiments, a biodegradable material can be used. In embodiments, poly(lactide-co-galactide) (PLGA) is a representative biodegradable material. In embodiments, any biodegradable material, including but not necessarily limited to biodegrable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In certain approaches, compositions of this disclosure, including the described systems, and cells modified using the described systems, are used for treatment of condition or disorder in an individual in need thereof. The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

In embodiments, a system of this disclosure is administered to an individual in a therapeutically effective amount. In embodiments, a therapeutically effective amount of a composition of this disclosure is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, cells modified according to this disclosure are administered to an individual in need thereof in a therapeutically effective amount.

In embodiments, the disclosure comprises providing a treatment to an individual in need thereof by introducing a therapeutically effective amount a composition of this disclosure, or modified cells as described herein to the individual, wherein the cells comprising the DNA insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure.

In embodiments, a described system of this disclosure is introduced into one or more prokaryotic or eukaryotic cells. In embodiments, the prokaryotic cells comprise or consist of gram positive, or gram negative bacteria. The bacteria may be non-pathogenic, or pathogenic. In embodiments, a described system is introduced into prokaryotic cells (e.g., bacterial or archaeal cells) in the context of a host, e.g., a human, animal, or plant host, e.g., the bacteria are a component of a host's microbiome or are an abnormal component of a microbiome, e.g., a pathogen. In some embodiments, delivery of a system described herein results in the stable formation of a recombinant microorganism. In some embodiments, a recombinant microorganism as generated by a system described herein results in the production of an enzyme or metabolite that can alter the health or metabolism of a host, e.g., a human host. In some embodiments, delivery of a system described herein results in the inactivation of virulence determinants of a microorganism, e.g., antibiotic resistance or toxin production. In some embodiments, delivery of a system described herein results in killing of the recipient cell. The system may kill some or all of the cells, or render the cells non-pathogenic and/or sensitive to one or more antibiotics. In embodiments, the bacteria are used as a component of a food or beverage product, including but not limited to fermented food and beverages, and dairy products. In embodiments, such bacteria comprise Lactic acid bacteria. In embodiments, selective delivery to a specific type of bacteria is used by way of a bacteriophage or packaged phagemids that can express all or some of the described components, but wherein the bacteriophage exhibits a specific tropism for a particular type of bacteria. In some embodiments, a delivery vehicle provides only partial specificity towards targeting particular cells, and additional specificity is provided by the choice of DNA sequence being targeted.

In embodiments, the described systems are introduced into eukaryotic cells. Such cells include but are not necessarily limited to animal cells, fungi such as yeasts, protists, algae, and plant cells.

In embodiments, the disclosure provides one or more cells, wherein DNA in the cells comprises at least one inserted DNA insertion template. The described cells may be any prokaryotic or eukaryotic cells. Accordingly, the disclosure also provides one or more cells that comprise an inserted DNA sequence.

In embodiments, the eukaryotic cells comprise animal cells, which may comprise mammalian or avian cells, or insect cells. In embodiments, the mammalian cells are human or non-human mammalian cells. In embodiments, compositions of this disclosure are administered to avian animals, or to a canine, a feline, an equine animal, or to cattle, including but not limited to dairy cattle.

In embodiments, the cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made.

In embodiments, the disclosure includes obtaining cells from an individual, modifying the cells ex vivo using a system as described herein, and reintroducing the cells or their progeny into the individual or a immunologically matched individual for prophylaxis and/or therapy of a condition, disease or disorder, or to treat an injury, trauma or anatomical defect. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, eukaryotic cells made according to this disclosure can be used to create transgenic, non-human organisms.

In embodiments, one or more modified cells according to this disclosure may be used to perform a gene-drive in a population of animals, including but not necessarily limited to insects.

In embodiments, the one or more cells into which a described system is introduced comprises a plant cell. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. Plant products made according to the disclosure are included.

In embodiments, the disclosure provides an article of manufacture, which may comprise a kit. In embodiments, the article of manufacture may comprise one or more cloning vectors. The one or more cloning vectors may encode any one or combination of proteins and polynucleotides described herein. The cloning vectors may be adapted to include, for example, a multiple cloning site (MCS), into which a sequence encoding any protein or polynucleotide, such as any desired targeting RNA, may be introduced. An article of manufacture may include one or more sealed containers that contain any of the aforementioned components, and may further comprise packaging and/or printed material. The printed material may provide information on the contents of the article, and may provide instructions or other indication of how the contents of the article may be used. In an embodiment, the printed material provides an indication of a disease or disorder that is to be treated using the contents of the article.

In embodiments, when polynucleotides are delivered, they may comprise modified polynucleotides or other modifications, such as phosphate backbone modifications, and modified nucleotides, such as nucleotide analogs. Suitable modifications and methods for making nucleic acid analogs are known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA).

The Examples of this disclosure are illustrated by Examples below and accompanying figures. While the disclosure has been described in conjunction with the detailed description and the Figures, this the Examples are intended to illustrate but not limit the scope of the invention. As illustrated by the Examples below, this disclosure provides in part an analysis of the molecular adaptations associated with CAST element evolution as well as mechanistic characteristics of the initial stages of CAST transposition. To obtain the data described in the Examples we analyzed interactions between the type I-F3b CRISPR-effector complex (referred to herein as Cascade) and TniQ. The I-F3 CAST elements fall in to two groups that show differences in architecture and regulation, (I-F3a and I-F3b) and a sister group that only uses guide RNA to target other mobile elements (see discussion) (Petassi et al., 2020). The disclosure provides a Cascade-TniQ structure from the I-F3b subtype revealing a full R-loop, describing the first steps in transposition initiation. The disclosure reveals multiple previously unappreciated roles for TniQ in licensing transposition, acting to distinguish guide-RNA categories, and as a platform for recruitment of downstream transposition components (akin to TnsD from prototypic Tn7). The disclosure also describes mechanisms allowing I-F3b CAST elements to develop PAM ambiguity for host immune surveillance escape and to tolerate diversification of attachment sites recognized by the system.

### Example 1

### Cryo-EM 3D Variability Analysis Reveals the Structure of the Full R-Loop and Dynamics of DNA Binding

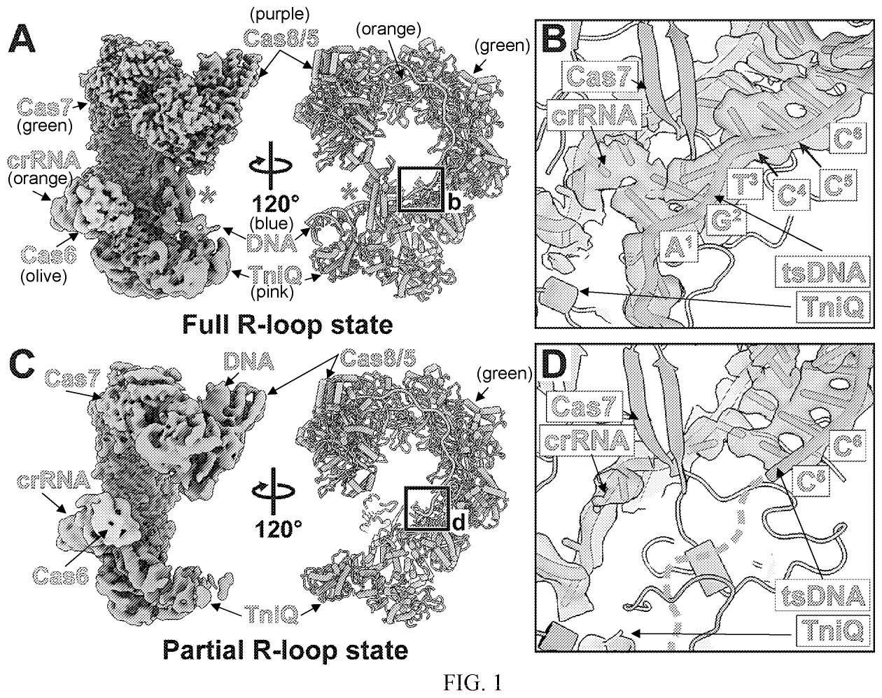

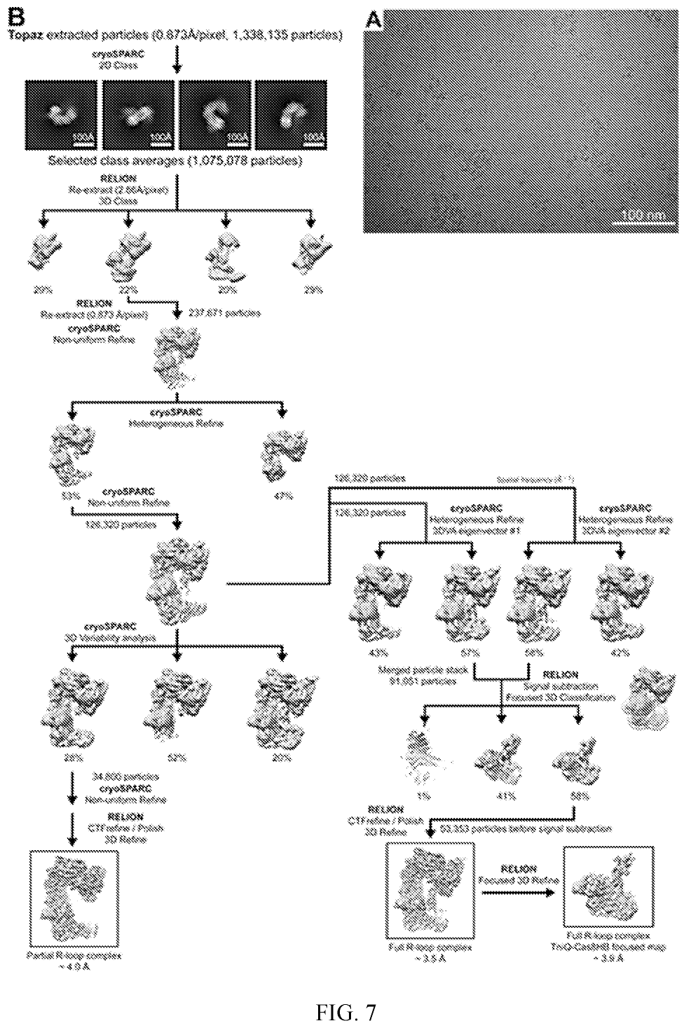

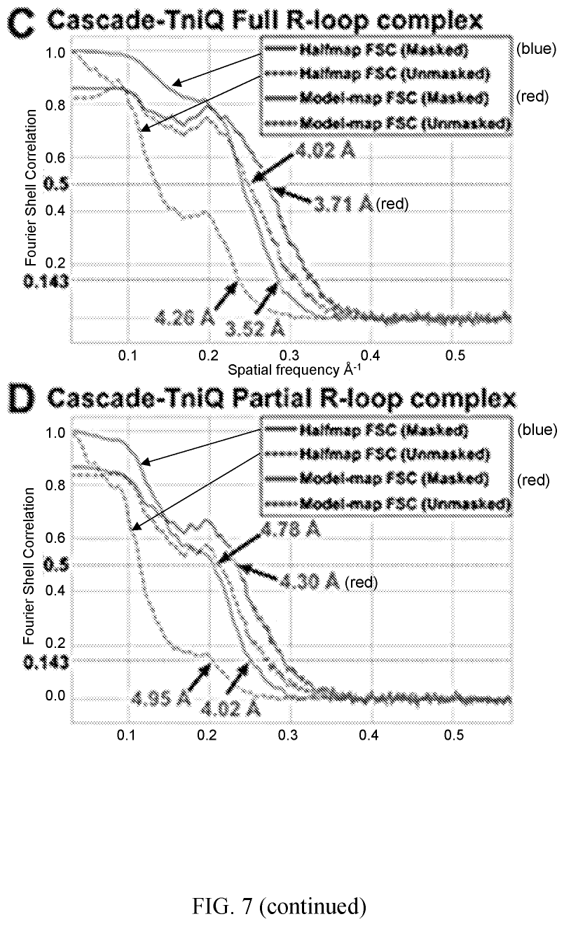

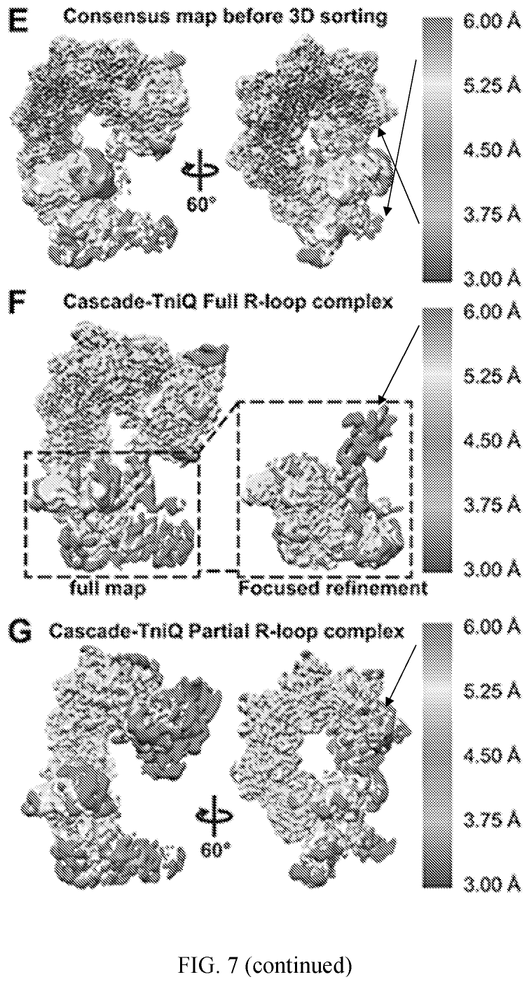

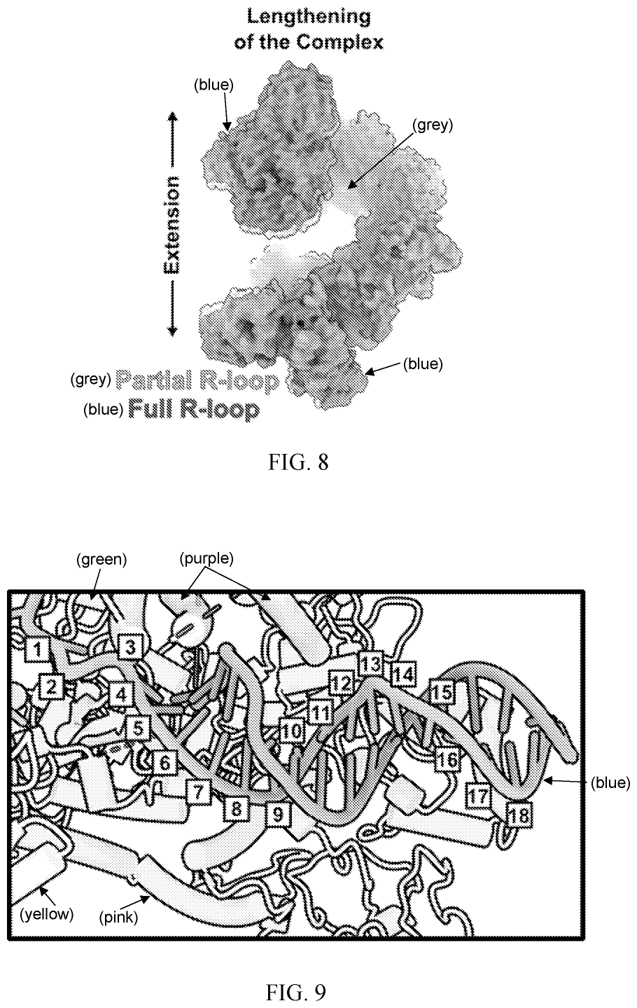

We used cryo-electron microscopy (cryo-EM) to structurally characterize the I-F3b element Tn6900 CRISPR-effector complex (a.k.a. Cascade, which includes proteins Cas6, Cas7, and Cas8/5) in association with the TniQ transposition protein. Consistent with previous reports (Halpin-Healy et al., 2020), cryo-EM 2D class averages revealed significant conformational heterogeneity, most prominently in the vicinity of TniQ (FIG. 1). The overall architecture of the I-F3b Cascade-TniQ complex closely matches previously published I-F3a cryo-EM structures (Halpin-Healy et al., 2020) (FIG. 1). As a result of the conformational dynamics present within the complex, the subsequent 3D reconstruction (overall assessed to be 3.2 A average resolution) contained comparatively weak TniQ density and variable local resolution throughout the structure (FIG. 7). 3D variability analysis (3DVA) (Punjani and Fleet, 2020) revealed the distinct structures within the sample: the dominant motion (i.e. separable using the first eigenvector) is the result of breathing motions in the complex separating partially-bound target DNA and fully engaged R-loop conformations (FIG. 1). Full R-loop formation is accompanied by a 900 rotation of the Cas8/5 fused helix bundle domain (HBD, FIG. 1A), missing from prior I-F3a structures (Halpin-Healy et al., 2020), and a slight expansion of the Cascade complex (FIG. 8). The observed conformational change is reminiscent of a similar conformational change in type I-F1 Cascade, in which the helical-bundle of Cas8f rotates roughly 180 degrees upon target DNA binding (Rollins et al., 2019). This conformational change we observe in the I-F3b particle population is tightly correlated with R-loop formation, which suggests that this transition is required prior to recruitment of downstream transposition factors (i.e., TnsC).

### Example 2

### R-Loop Structure Reveals how TniQ Creates a Platform for Recruitment of Core Transposition Proteins.

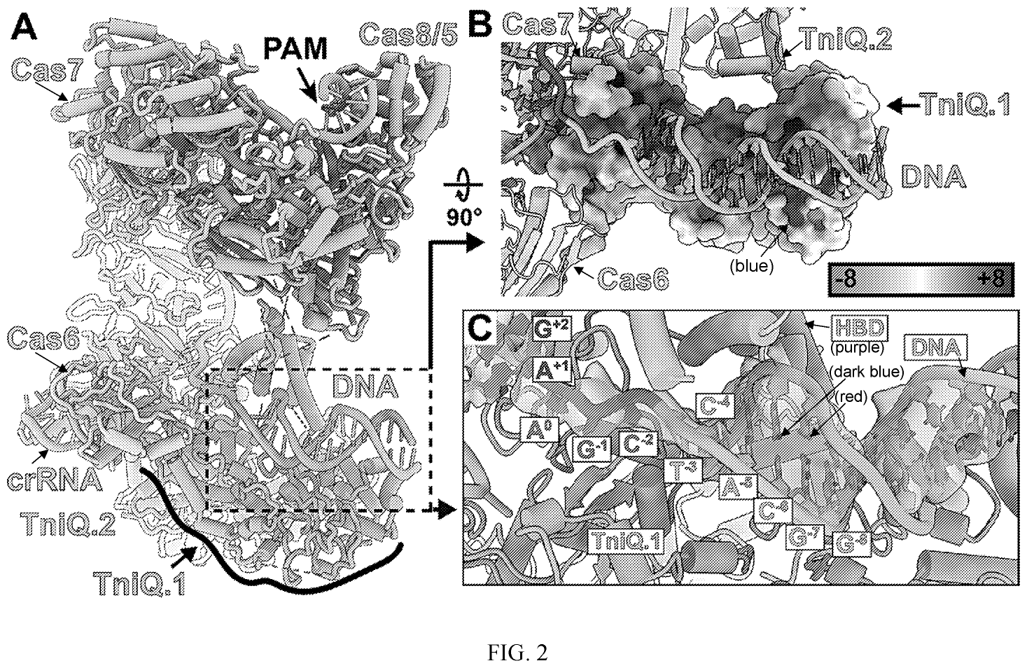

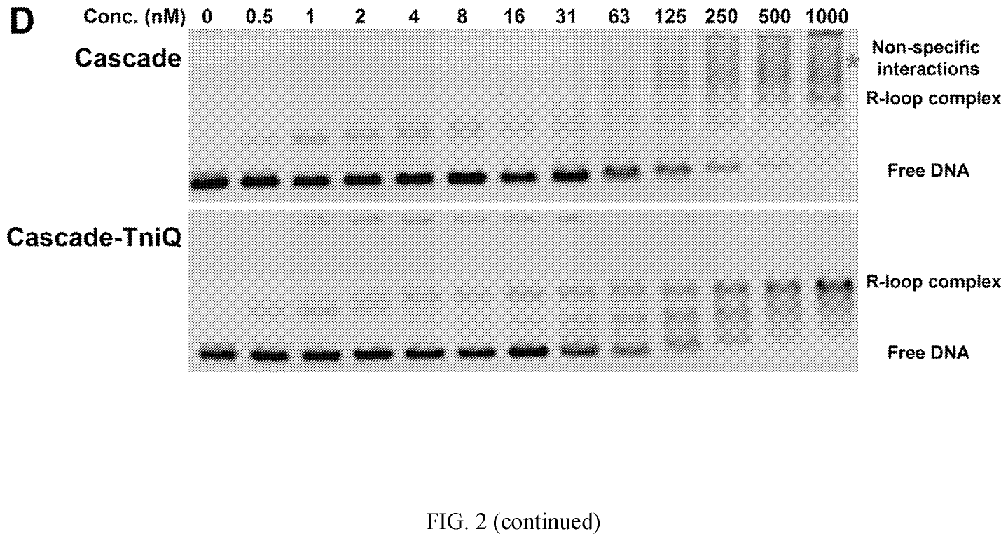

Clustering particle images according to 3DVA eigenvectors resulted in a 3.3 Å resolution cryo-EM reconstruction of the full R-loop (including PAM-distal duplex DNA), sufficient for atomic modeling (FIG. 2A). This structure implies that Cascade-TniQ is sufficient to stabilize the full R-loop and does not require additional transposition components (i.e. TnsABC) or host factors for productive protospacer engagement. PAM-distal duplex DNA was not observed in previously determined I-F1 CRISPR-Cas structures (Rollins et al., 2019), suggesting that, in this case, TniQ may serve as an additional platform in this system for stabilizing the distal end of the R-loop preparing the DNA substrate for transposition initiation. Although TniQ is associated with Cascade as a homo-dimer, only one monomer of TniQ associates with double-stranded DNA (dsDNA), which follows a path roughly parallel to the dimerization interface of TniQ (FIG. 2B). The TniQ surface in contact with DNA is highly basic (FIG. 2B) and has a footprint that spans 18 base pairs (FIG. 9). We also observed distortion in the PAM distal DNA corresponding to a slight lengthening and duplex DNA unwinding (FIG. 2C). This distortion corresponds to a maximum helical pitch of 38A (compared with 34A in ideal B-form DNA) (FIG. 2C) and appears to be primarily driven by the re-joining of target and non-target DNA strands mediated by the Cas8/5 helical bundle domain and TniQ (FIG. 2B-C). The DNA distortion is further accompanied by unwinding of approximately 3 base pairs past the expected spacer region (FIG. 2B-C).

Without intending to be constrained by any particular view, the disclosure provides a model in which the Cascade-TniQ complex presents an ideal substrate for TnsC binding by stabilizing underwound, elongated DNA cooperatively along with the Cas8/5 helix bundle domain (FIG. 2A-D). Based on these structural observations, we reasoned that TniQ may have a second, previously unappreciated role in stabilizing R-loop formation. In order to analyze this, we probed the extent of R-loop formation comparing purified Cascade and Cascade-TniQ complexes using electrophoretic mobility shift assays (EMSAs)(FIG. 2D). Cascade without TniQ was unable to form a defined complex with the target DNA and had significant smeared product at higher concentrations (* on FIG. 2D, top panel). On the other hand, Cascade-TniQ complex formed a substantial amount of partial R-loop complex at low concentrations (FIG. 2D, bottom), which progressed to full R-loop complex at higher concentrations (FIG. 2D, bottom). Consistently, the smear of bands found at high concentrations of Cascade was not observed when TniQ was added, indicating that TniQ is essential to transform bound DNA substrate into partial and full R-loop products.

### Example 3

### Tniq is Responsible for Guide-RNA Category Discrimination

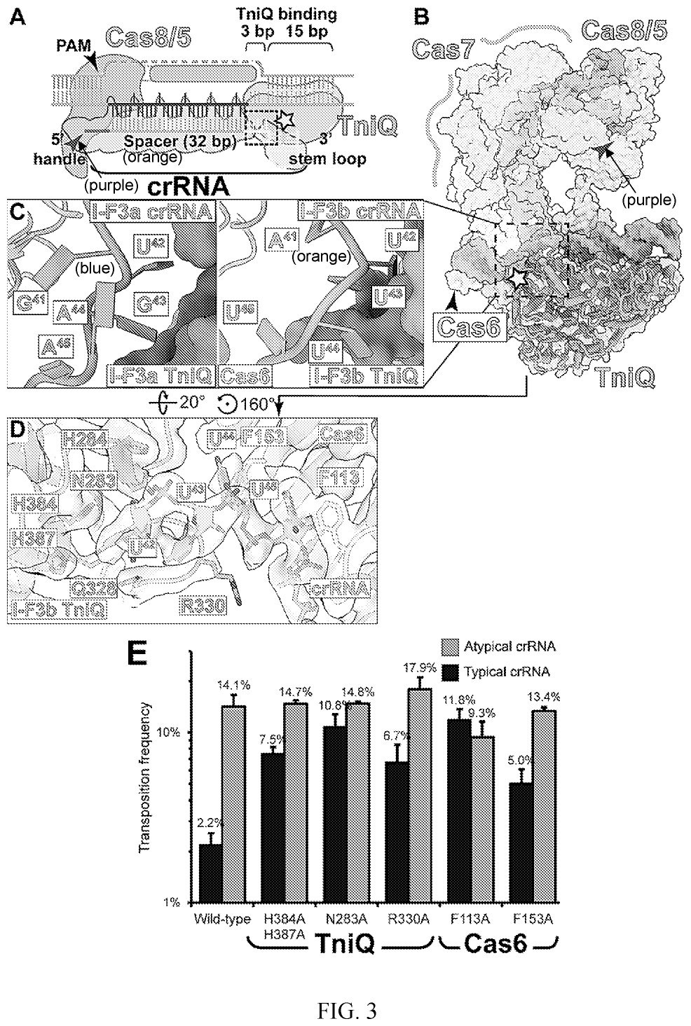

Type I-F Cascade engages with a 60-nt crRNA, including a 32-nt spacer derived sequence (orange, FIG. 3A&B) flanked by 8-nt 5′ handle (bound by Cas8/5, indicated with purple arrow, FIG. 3A&B) and a 20-nt 3′ stem loop recognized by Cas6 (indicated with a star, FIG. 3A&B). Closer inspection of the region adjacent to the 3′ stem loop RNA reveals significant differences between the I-F3a (PDB: 6PIF, FIG. 3C left) and I-F3b structures (this study, FIG. 3C right). In the I-F3a structure, the crRNA is too distant to make substantial interactions with TniQ (FIG. 3C, left). However, the crRNA in the I-F3b structure reported here adopts a different path, with two bases (U42 and U44) sufficiently close to interact with TniQ (FIG. 3C, right). This suggests that the molecular mechanisms for discriminating between different guide-RNA sequences (i.e. typical versus atypical, as reported previously (Petassi et al., 2020)) might originate from TniQ.

Although many of the TniQ residues in the vicinity of these base-pairs are generally basic (FIG. 3D), we are unable to identify RNA sequence-specific interactions that might explain the substantial activity difference between typical and atypical crRNA (Petassi et al., 2020). Nevertheless, alanine mutation of proximal residues in TniQ (N283, R330, H384, and H387) and Cas6 (F113 and F153) results in a partial (F153A, R330A, and H384A,H387A) or full restoration (N283A and F113A) of transposition activity of typical crRNA relative to atypical (FIG. 3E), indicating defects in the mechanism allowing regulation of guide-RNA preference. This demonstrates that TniQ plays an important role in I-F3b targeting preferences (Petassi et al., 2020), and highlights TniQ as the central component that is capable of modulating CAST guide-RNA dependent targeting.

### Example 4

### Comparative Analysis of I-F3b PAM and I-F1 PAM Requirements for Transposition and Interference.

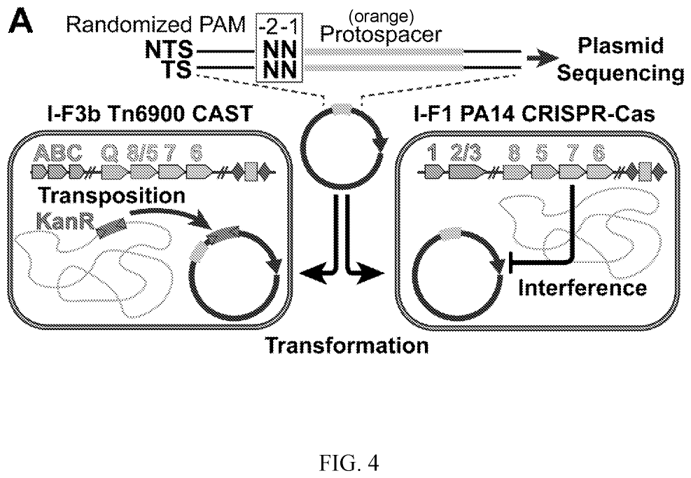

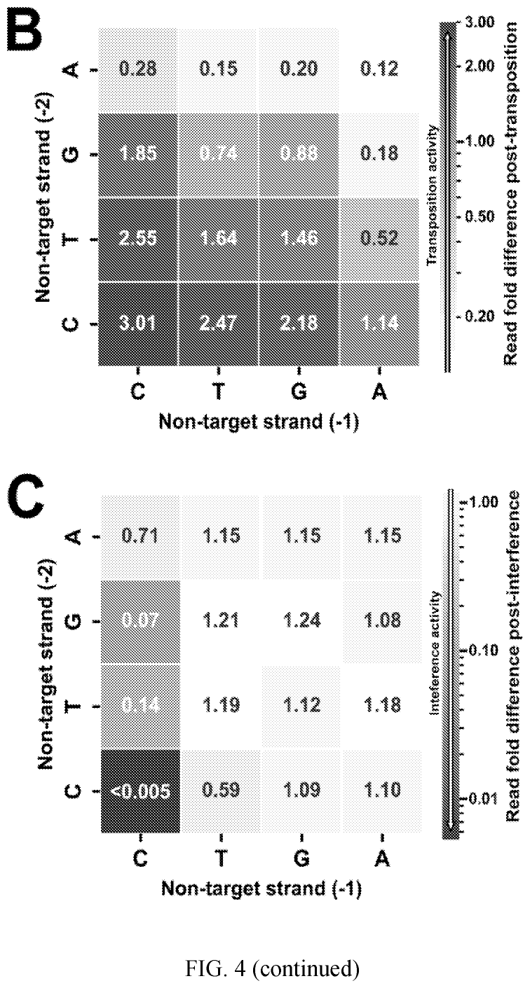

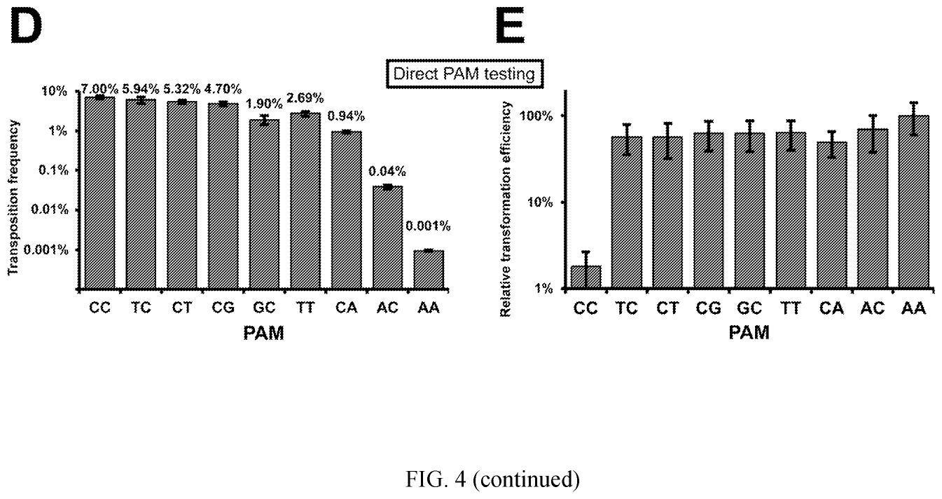

Canonical I-F1 systems require a non-target strand CC PAM for interference activity (Rollins et al., 2015). In contrast, previous work has highlighted expanded PAM flexibility with I-F3 CAST systems compared to the canonical I-F1 CRISPR-Cas defense systems (Klompe et al., 2022; Klompe et al., 2019; Petassi et al., 2020; Rybarski et al., 2021; Vo et al., 2021; Wimmer et al., 2022; Yang et al., 2021). As part of our comparative analysis, we performed an unbiased PAM screen utilizing a target plasmid pool randomized at the PAM sequence (“−2” and “−1” positions, FIG. 4A) associated with a plasmid-encodedffs target sequence (FIG. 4A). The target plasmid with the randomized PAM was then transformed into a cell population expressing the I-F3b Tn6900 CAST system or the 1-F1 P. aeruginosa PA14 CRISPR-Cas defense system (FIG. 4A). The transposition potential of all possible PAM combinations (4²) was assessed by looking for PAM sequence enrichment in the population of plasmids that were targeted for transposition (FIG. 4B) or interference (FIG. 4C) when compared to input plasmid population.

The I-F1 interference system had a strong preference for CC, which was completely depleted from the pool (i.e. “<0.005” in FIG. 4C). More generally, I-F1 interference activity depleted plasmids with a C in the −1 position (FIG. 4C). By contrast, in the I-F3b transposition screen, we observe an overall loosening of PAM requirements (FIG. 4B). While the CC PAM did show a 3-fold enrichment as a transposition target (FIG. 4B), all of the PAMs combinations in the pool were used as transposition targets. (FIG. 4B). To further validate these results, a subset of PAM sequences were individually constructed and tested in the mate-out transposition assay for I-F3b CAST (FIG. 4D) or interference assay for I-F1 CRISPR-Cas (FIG. 4E). While the CC PAM was efficiently used as a target for transposition, many additional PAM combinations could be recognized as transposition targets, often only differing by a few percent with their transposition efficiency. This was markedly different than the result found with the interference assay with an almost two-order of magnitude difference being found between the appropriate CC PAM and any of the other PAM combinations (FIG. 4D&E).

### Example 5

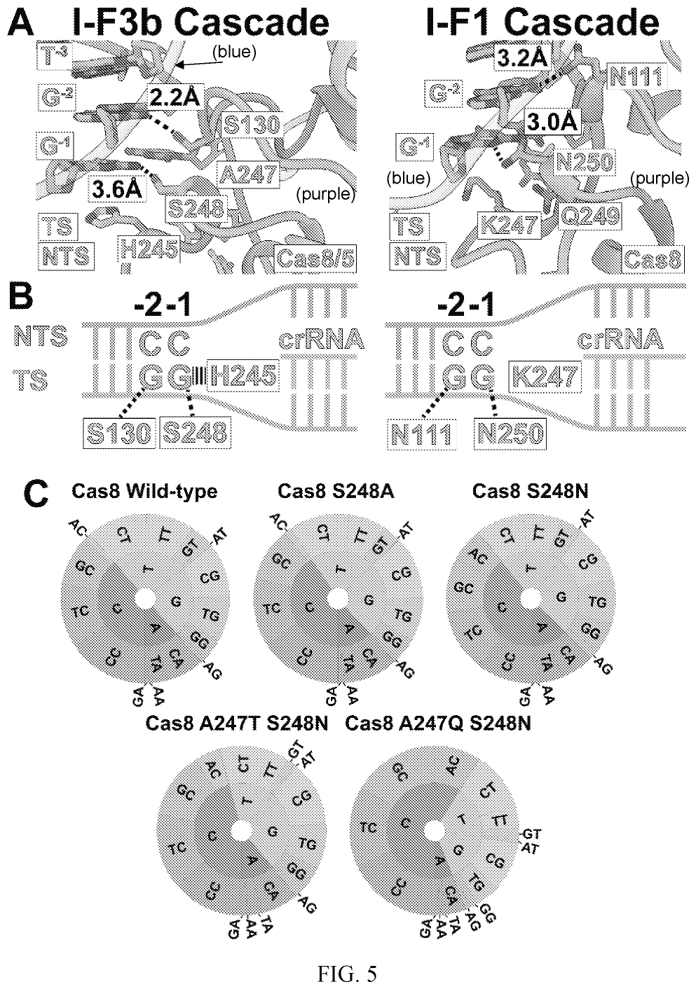

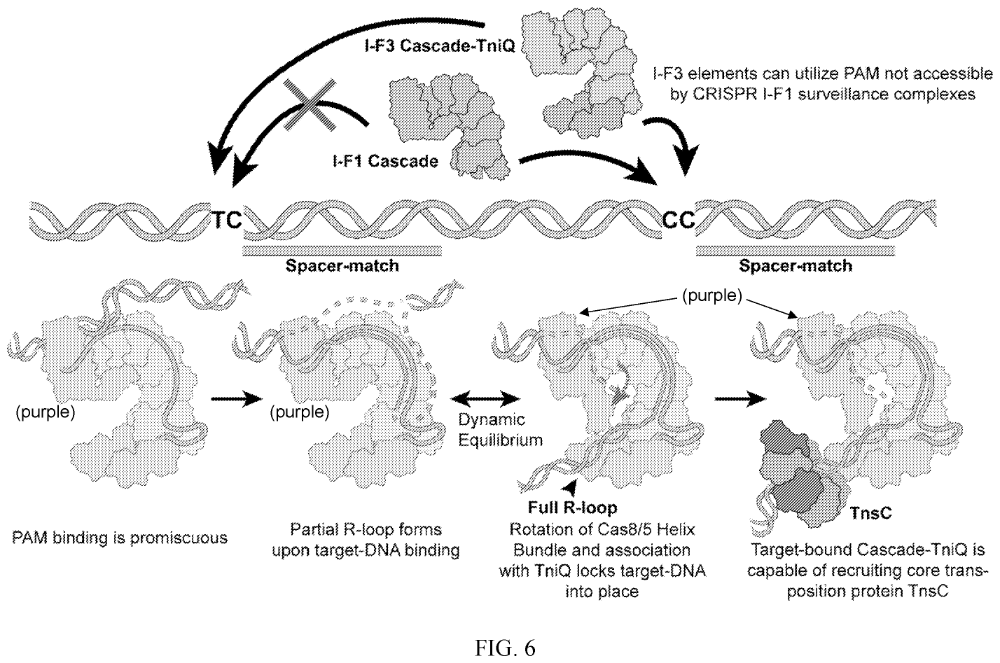

Molecular basis of I-F3 CAST PAM promiscuity and specificity Inspection of structures representing the I-F1 CRISPR-Cas associated Cascade complex (PDB: 6NE0) (Rollins et al., 2019) and the I-F3b CAST-associated Cascade-TniQ complex reveal candidate residues that could possibly explain PAM sequence preferences (FIG. 5A). Notably, serines S248 (3.58A) and S130 (2.2A) are within hydrogen-bonding distance of the −1 and −2 PAM bases, respectively, in the I-F3b CAST structure (FIG. 5A). In comparison, the −1 and −2 PAM bases in the I-F1 CRISPR-Cas structure are interacting with N250 and N111, respectively (FIG. 5B). While serine residues would be largely agnostic to the specific PAM sequence, asparagine residues (having both hydrogen bond donors and acceptor groups) would be able to make base-specific interactions. To explore the level of PAM specificity in the I-F3b system, we made changes in the S248 residue predicted to interact with the −1 position of the PAM (FIG. 4A). Interestingly, neither the S248N nor the S248A could reestablish any substantial level of PAM discrimination (FIG. 5C). This indicates that PAM specificity is strongly dependent on other energetic effects besides simply contacts (i.e. hydrogen bonding) with DNA.

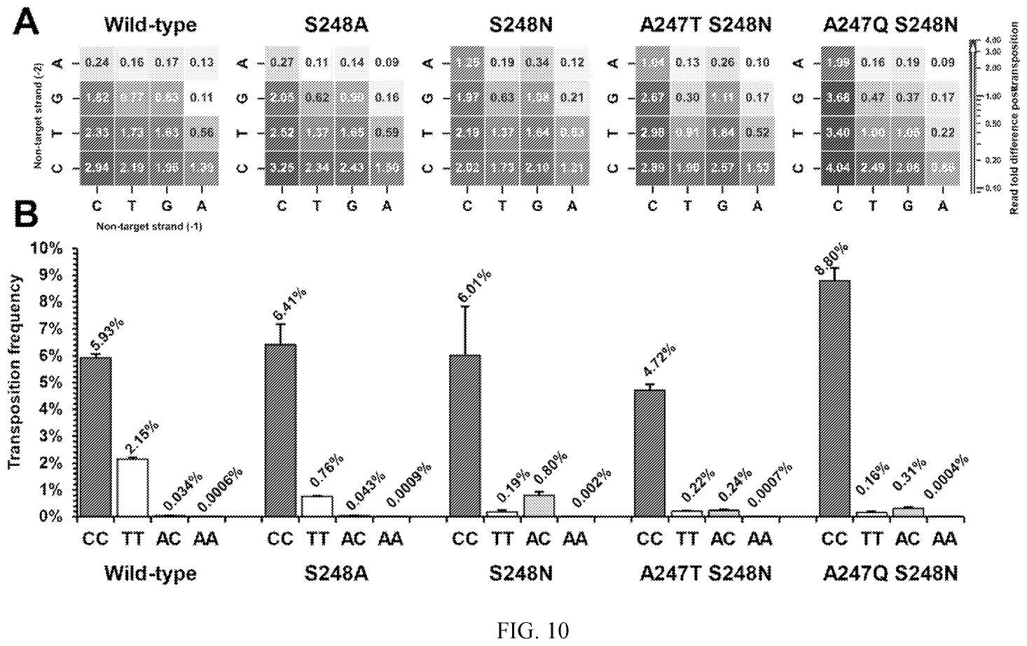

To address if the local environment that positions the S248 residue was also important for PAM discrimination, we examined mutants with the A247T+S248N and A247Q+S248N changes. The changes involving the residue S248N contacting the PAM and an adjacent residue increased sequence specificity at the predicted −1 position to bias transposition to PAMs with a C at the −1 position (FIG. 5B and FIG. 10A). Unlike wild type Cas8/5 or the other mutants we examined, over half of the transposition events in the pool with the A247Q S248N change had the PAM with C-1 (FIG. 5C).

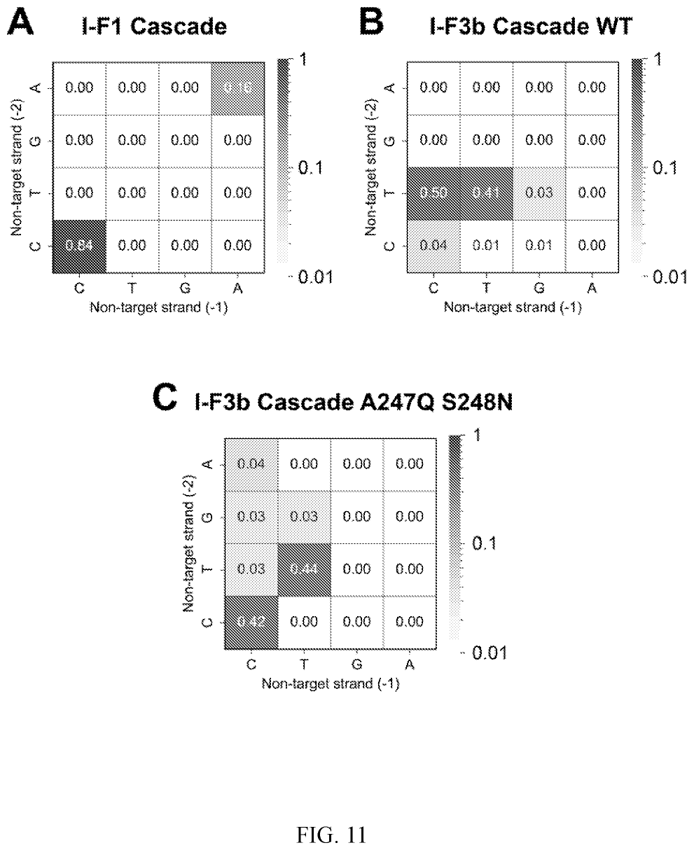

Our results indicate that the PAM binding region of Cas8/5 has likely been subjected to extensive selection for PAM ambiguity. Single amino acid changes at residues that directly contact the PAM are not sufficient to reestablish PAM CC selectivity. However, more extensive changes involving multiple residues in the PAM binding pocket can start to reestablish the −1 C preferences suggesting rational design methods focused on the PAM binding pocket could reconfigure PAM selectivity. Computational modeling of the I-F1, I-F3b, and I-F3b mutant (A247Q+S248N) generally captures the experimental trends described here (FIG. 11), and supports design strategies that incorporate both flexible backbone modeling as well as consideration of the full PAM-binding pocket. We suggest that two important factors have selected for PAM ambiguity in I-F3 transposition systems: host immune surveillance escape and diversification of attachment sites recognized by the system.

It will be recognized from the Examples that the present disclosure reveals co-option of type I-F cascade for guide RNA-directed transposition involves extensive reconfiguration of the control of the CRISPR-Cas effector repurposed by TniQ and downstream components for transposon targeting. The provided high-resolution structure reveals a series of new interactions between TniQ and downstream DNA and with the Cas8/5 helix bundle domain (FIGS. 1 and 2). These contacts facilitate new functions we identify with TniQ for stabilizing the cascade complex into a stable full R-loop, an important step for licensing a target for transposition in these systems (FIGS. 1 and 2). Additionally, the TniQ-DNA contacts identified herein allow a DNA distortion predicted to help accommodate TnsC-mediated transposase recruitment based on previous work with prototypic Tn7 (FIG. 3). We show that TniQ acts to mediate guide RNA selection, sorting typical and atypical guide RNAs to regulate target choice in the system (FIG. 3). Direct comparison between PAM usage in I-F3 transposition and I-F1 interference systems indicates the extent of PAM ambiguity in the transposition systems (FIG. 4) and that extensive structural adaptations contribute to the process (FIG. 5). These results support a model where control features normally used by Cascade are now licensed by TniQ either directly or with the collaboration of other transposition components.

### Tniq is a Central Cascade Regulator

TniQ/TnsD proteins have adapted to a variety of fixed att sites in bacteria and to programmable att sites (Hsieh and Peters, 2021; Petassi et al., 2020; Peters et al., 2017). Prototypic Tn7 distinguishes between target sites using either of two proteins that function in parallel targeting pathways: one allowing sequence-specific DNA-binding (TnsD/TniQ) or one recognizing specialized features of DNA replication found with mobile plasmids (TnsE) (Mitra et al., 2010; Parks et al., 2009; Shi et al., 2015). A sister group with the I-F3 CAST elements and two independently coopted Type I-B CAST elements use a hybrid approach half-way between prototypic Tn7 and I-F CAST elements, where pathway choice occurs via association with either a sequence-specific TnsD/TniQ protein or a Cascade-TniQ complex (Klompe et al., 2022; Petassi et al., 2020; Saito et al., 2021). Without intending to be bound by any particular theory it is considered that in these cases TnsC is the master regulator that is capable of recognizing different insertion sites by preferentially associating with particular TnsD/TniQ or Cascade-TniQ. This decision role for TnsC is not compatible for transposition systems with a single dedicated TniQ protein and complex regulatory behavior, as with the I-F3 CAST family studied here and type V-K CAST elements.

The disclosure indicates that in the I-F3 CAST systems, TniQ actively cooperates with Cascade to regulate transposition by controlling R-loop formation in a manner that senses guide-RNA categories. This adds important insight into the function of TniQ, which previously was believed to function simply as a physical connector between target-site and core transposition components (Halpin-Healy et al., 2020). Not only does the disclosure provide a strong mechanistic basis for understanding previous studies highlighting complex regulatory behavior in I-F3b CAST (Petassi et al., 2020) and related elements (Saito et al., 2021; Shan-Chi Hsieh, 2021), but also reveals an evolutionarily conserved role for the TniQ/TnsD protein superfamily in facilitating decision making at attachment sites.

### Cascade-TniQ Distorts the Target, a Mechanism Known to Drive Recruitment of Transposition Proteins

TniQ and the helix-bundle domain of Cas8/5 bind and distort DNA downstream from the protospacer (FIG. 3) in a manner that resembles the target DNA distortion produced by Tn7 TnsD (Kuduvalli et al., 2001; Mitra et al., 2010). This DNA distortion has been implicated in recruitment of Tn7 TnsC regulator, as Tn7 TnsC will direct transposition to altered structure triplex DNA substrates alone (Rao et al., 2000). This model is further supported by a cryo-EM structure revealing that Tn7 TnsC specifically loads adjacent to mismatched bubble DNA substrates (Shen et al., 2022). With type V-K CAST elements, TnsC loading seems to occur spontaneously using a search filament to identify TniQ associated with the effector complex (Park et al., 2021), a behavior more reminiscent of the MuB AAA+ protein from bacteriophage Mu (Mizuno et al., 2013). The mechanistic implications of this disclosure indicate that activation of transposition with each CAST element family may have evolved different strategies to solve similar problems.

### PAM Ambiguity is a Mechanistic Adaptation of I-F3 Systems Allowing Att Site Drift Tolerance and Privatizes Guide RNAs

The disclosure indicates that PAM flexibility is important for two reasons in the I-F3 CAST transposition systems: 1. to maintain a fixed attachment site recognizable in diverse host chromosomes and 2. for privatizing attachment sites inaccessible to the host interference system. The described results extend generally to naturally occurring populations; only a small percentage (3.4%, 8/235) of the I-F3bffs att sites identified in genome sequences utilize a CC PAM with all other examples using TC. Overall, the CC PAM is only used 4.4 percent (33/757) of the time across all of the I-F3 insertion sites identified in bacterial genomes suggesting this is a general attribute of this family of CAST elements. Given that canonical I-F1 CRISPR-Cas systems often reside in the same host as I-F3 elements, privatization by PAM ambiguity as indicated here (FIG. 4E) is likely another important mechanism for I-F3 elements that have guide RNAs to recognize chromosomal attachment sites (Petassi et al., 2020).

The disclosure includes rational modification of PAM for use in orthogonally functioning, precise CAST elements. The disclosure indicates that extensive rearrangements were under selection to allow PAM ambiguity in the I-F3 systems, because multiple amino acid changes were needed to bias PAM selection to the CC PAM used by the canonical I-F1 system (FIG. 5). Our computational simulations suggests that accurate modelling should incorporate flexible backbone modeling of the entire PAM binding site, not just the positions directly interacting with the PAM motif (FIG. 11).

Together, the described results indicate that multiple adaptive changes in both the CRISPR-effector (i.e. Cascade) and the associated TniQ work together to enhance CAST survival and facilitate transposon distribution. TniQ's ability to distinguish between categories of insertion sites and the tight control TniQ exhibits over the transposition process is an example of the dynamic molecular mechanisms utilized to escalate the host-pathogen arms race. Rational modification of CAST elements is included in this disclosure to enhance the functionality of CAST elements in genome-editing applications, but has been previously limited due to a lack of mechanistic and structural understanding of CAST transposition. The present disclosure expands mechanistic understanding of I-F3b CAST elements and places functional understanding of TniQ into context. It also introduces a collection of previous unknown protein, DNA, and RNA interactions that can be further engineered for adapting these systems for genome engineering.

The following references relate to Examples 1-5. This reference listing is not an indication that any reference is material to patentability.

### REFERENCES

Halpin-Healy, T. S., Klompe, S. E., Sternberg, S. H., and Fernindez, I. S. (2020). Structural basis of DNA targeting by a transposon-encoded CRISPR-Cas system. Nature 577, 271-274.
Hsieh, S.-C., and Peters, J. E. (2021). Tn7-CRISPR-Cas12K elements manage pathway choice using truncated repeat-spacer units to target tRNA attachment sites. bioRxiv.
Jia, N., Xie, W., de la Cruz, M. J., Eng, E. T., and Patel, D. J. (2020). Structure-function insights into the initial step of DNA integration by a CRISPR-Cas-Transposon complex. Cell Res 30, 182-184.
   Klompe, S. E., Jaber, N., Beh, L. Y., Mohabir, J. T., Bernheim, A., and Sternberg, S. H. (2022). Evolutionary and mechanistic diversity of Type I-F CRISPR-associated transposons. Mol Cell.
Klompe, S. E., Vo, P. L. H., Halpin-Healy, T. S., and Sternberg, S. H. (2019). Transposon-encoded CRISPR-Cas systems direct RNA-guided DNA integration. Nature 571, 219-225.
Kuduvalli, P. N., Rao, J. E., and Craig, N. L. (2001). Target DNA structure plays a critical role in Tn7 transposition. EMBO J 20, 924-932.
Li, Z., Zhang, H., Xiao, R., and Chang, L. (2020). Cryo-EM structure of a type IF CRISPR RNA guided surveillance complex bound to transposition protein TniQ. Cell research 30, 179-181.
Mitra, R., McKenzie, G. J., Yi, L., Lee, C. A., and Craig, N. L. (2010). Characterization of the TnsD-attTn7 complex that promotes site-specific insertion of Tn7. Mob DNA 1, 18.
Mizuno, N., Dramicanin, M., Mizuuchi, M., Adam, J., Wang, Y., Han, Y. W., Yang, W., Steven, A. C., Mizuuchi, K., and Ramon-Maiques, S. (2013). MuB is an AAA+ ATPase that forms helical filaments to control target selection for DNA transposition. Proc Natl Acad Sci USA 110, E2441-2450.
Park, J. U., Tsai, A. W., Mehrotra, E., Petassi, M. T., Hsieh, S. C., Ke, A., Peters, J. E., and Kellogg, E. H. (2021). Structural basis for target site selection in RNA-guided DNA transposition systems. Science 373, 768-774.
Parks, A. R., Li, Z., Shi, Q., Owens, R. M., Jin, M. M., and Peters, J. E. (2009). Transposition into replicating DNA occurs through interaction with the processivity factor. Cell 138, 685-695.
Petassi, M. T., Hsieh, S. C., and Peters, J. E. (2020). Guide RNA Categorization Enables Target Site Choice in Tn7-CRISPR-Cas Transposons. Cell 183, 1757-1771 e1718.
Peters, J. E. (2015). Tn7. In Mobile DNA III, L. Craig Nancy, P. Rice, A. Lambowitz, M. Gellert, and S. B. Sandmeyer, eds. (Washington DC: ASM Press), p. In Press.
Peters, J. E. (2019). Targeted transposition with Tn7 elements: safe sites, mobile plasmids, CRISPR/Cas and beyond. Mol Microbiol 112, 1635-1644.
Peters, J. E., Makarova, K. S., Shmakov, S., and Koonin, E. V. (2017). Recruitment of CRISPR-Cas systems by Tn7-like transposons. Proc Natl Acad Sci USA 114, E7358-E7366.
Punjani, A., and Fleet, D. J. (2020). 3D Variability Analysis: Directly resolving continuous flexibility and discrete heterogeneity from single particle cryo-EM images. Cold Spring Harbor Laboratory.
Rao, J. E., Miller, P. S., and Craig, N. L. (2000). Recognition of triple-helical DNA structures by transposon Tn7. Proc Natl Acad Sci USA 97, 3936-3941.
Rollins, M. F., Chowdhury, S., Carter, J., Golden, S. M., Miettinen, H. M., Santiago-Frangos, A., Faith, D., Lawrence, C. M., Lander, G. C., and Wiedenheft, B. (2019). Structure Reveals a Mechanism of CRISPR-RNA-Guided Nuclease Recruitment and Anti-CRISPR Viral Mimicry. Mol Cell 74, 132-142 e135.
Rollins, M. F., Schuman, J. T., Paulus, K., Bukhari, H. S., and Wiedenheft, B. (2015). Mechanism of foreign DNA recognition by a CRISPR RNA-guided surveillance complex from Pseudomonas aeruginosa. Nucleic Acids Res 43, 2216-2222.
Rybarski, J. R., Hu, K., Hill, A. M., Wilke, C. O., and Finkelstein, I. J. (2021). Metagenomic discovery of CRISPR-associated transposons. Proc Natl Acad Sci USA 118.
Saito, M., Ladha, A., Strecker, J., Faure, G., Neumann, E., Altae-Tran, H., Macrae, R. K., and Zhang, F. (2021). Dual modes of CRISPR-associated transposon homing. Cell.
Shan-Chi Hsieh, J. E. P. (2021). Tn7-CRISPR-Cas12K elements manage pathway choice using truncated repeat-spacer units to target tRNA attachment sites. bioRxiv.
Shen, Y., Gomez-Blanco, J., Petassi, M. T., Peters, J. E., Ortega, J., and Guarne, A. (2022). Structural basis for DNA targeting by the Tn7 transposon. Nature Structural & Molecular Biology 29, 143-151.
Shi, Q., Straus, M. R., Caron, J. J., Wang, H., Chung, Y. S., Guarne, A., and Peters, J. E. (2015). Conformational toggling controls target site choice for the heteromeric transposase element Tn7. Nucleic Acids Res.
Vo, P. L. H., Ronda, C., Klompe, S. E., Chen, E. E., Acree, C., Wang, H. H., and Sternberg, S. H. (2021). CRISPR RNA-guided integrases for high-efficiency, multiplexed bacterial genome engineering. Nat Biotechnol 39, 480-489.
Wang, B., Xu, W., and Yang, H. (2020). Structural basis of a Tn7-like transposase recruitment and DNA loading to CRISPR-Cas surveillance complex. Cell research 30, 185-187.
Wimmer, F., Mougiakos, I., Englert, F., and Beisel, C. L. (2022). Rapid cell-free characterization of multi-subunit CRISPR effectors and transposons. Mol Cell.
Yang, S., Zhang, Y., Xu, J., Zhang, J., Zhang, J., Yang, J., Jiang, Y., and Yang, S. (2021). Orthogonal CRISPR-associated transposases for parallel and multiplexed chromosomal integration. Nucleic Acids Res 49, 10192-10202.

### Example 6

This Example provides a description of the materials and methods used to produce the results in the foregoing Examples.

### Protein Purification

Expression plasmid sets were transformed into E. coli T7 Express cells (New England Biolabs). For Cascade-TniQ for CryoEM, plasmid set was pOPO066 (pETDuet-1-cas8/5-cas7), pOPO097 (pACYCDuet-1-cas6-cas7), pOPO127 (pCOLADuet-1-His6-tniQ), and pGS100 (pCDFDuet_crRNA-ffsx6). For Cascade-TniQ for EMSA, plasmid set was pOPO066 (pETDuet-1-cas8/5-cas7), pOPO097 (pACYCDuet-1-cas6-cas7), pOPO127 (pCOLADuet-1-His6-tniQ), and pMTP1277 (pCDFDuet_crRNA-ffsx6(alt)). For Cascade without TniQ for EMSA, plasmid set was pOPO065 (pETDuet-1-His6-cas8/5-cas7), pOPO097 (pACYCDuet-1-cas6-cas7), and pMTP1277 (pCDFDuet_crRNA-ffsx6(alt)). Cells were grown in LB with appropriate antibiotics at 37° C. to O.D.₆₀₀ 0.8 and induced overnight at 16° C. with 0.4 mM IPTG. Cells were pelleted, resuspended in lysis buffer (500 mM NaCl 25 mM HEPES pH 7.5 10% glycerol 5 mM DTT) plus 1 mM PMSF, and lysed by sonication. After sonication cells were centrifuged at 12,000 rpm for 45 min and the supernatant was collected and imidazole was added to a final concentration of 20 mM. The supernatant was loaded to 2 mL Ni-NTA resin (Thermofisher). The Ni-NTA resin was washed with 150 mL of lysis buffer with graded increases of imidazole to 50 mM. Complexes were eluted from the Ni-NTA resin with lysis buffer plus 300 mM imidazole. Eluted cascade complexes were then purified with anion exchange chromatography (MonoQ 5/50GL cytiva). Peak fractions were collected and snap-frozen in liquid nitrogen for later use.

### Dna Substrate Preparation

Bubbled dsDNA substrate for CryoEM was created by heating 4 oligonucleotides to 95° C. for 10 min in duplex buffer (30 mM HEPES, pH 7.5; 100 mM potassium acetate) followed by slow cooling. Annealed DNA was ligated with T4 ligase (ThemoFischer), ran on a 12% UREA-PAGE gel, and successfully ligated bands were cut and extracted from the gel. Purified ssDNA was then reannealed in duplex buffer, run on a 1% agarose gel to remove free ssDNA, and purified using GeneJet gel extraction kit (ThermoFischer).

### Electrophoretic Mobility Shift Assay

The perfectly matched and wild-type (wt) ffs protospacers were cloned into pJET vectors (ThermoFischer). 214 bp DNA targets were PCR amplified from their corresponding pJET vectors using fluorescently labeled primers. The perfectly matched and wt target DNAs were mixed at a 1:1 molar ratio and diluted to a final concentration of 0.5 nM each in EMSA buffer (100 mM KCl, 5% glycerol, 5 mM MgCl2, 2 mM β-mercaptoethanol). Titrations of Cascade or Cascade-TniQ were mixed with the diluted DNA targets and incubated at 37° C. for 15 minutes. 20 μL of DNA binding reactions were run on a 1% agarose TBE gel for 1 hour and 15 minutes at 60 V at 4° C. The gel was imaged with an Amersham Typhoon Biomolecular Imager (GE Healthcare Life Sciences) using corresponding filters for either perfectly matched DNA or wt DNA, and analyzed using ImageQuant 1D version 8.2 (GE Healthcare Life Sciences). For FIG. 2D, gel image from perfectly matched DNA substrate was used.

### Cryo-EM Sample Preparation and Imaging.

For the Cascade-TniQ with atypical crRNA, purified Cascade-TniQ sample was supplemented with 1.2-fold molar excess of target DNA with 32 base artificial bubble (see above). Then the Cascade-TniQ in the solution was diluted to 2 μM (˜0.9 mg/mL) making the final buffer composition as follows: 25 mM HEPES pH 7.5, 150 mM NaCl. The sample was incubated for 30 minutes on ice before being vitrified using the Mark IV Vitrobot (ThermoFisher) set to 4° C. and 100% humidity. 4 μL of the reconstituted Cascade-TniQ-DNA sample is loaded on the QuantiFoil Cu 1.2/1.3 grids (Quantifoil) that was freshly glow discharged using PELCO easiGlow (Ted Pella). Then the grids were immediately blotted for 6 seconds with blot force 6, followed by vitrification in the slurry of liquid ethane cooled with liquid nitrogen. The grids were first screened using Talos Arctica (ThermoFisher) operating at 200 kV, equipped with K3 direct electron detector (Gatan) and BioQuantum energy filter. Ice thickness, number of particles per image, and number of good squares are assessed to find the best grid for data collection. The chosen grid was imaged using Titan Krios G3 (ThermoFisher) operated at 300 kV, also equipped with K3 detector (Gatan) and BioQuantum energy filter (Gatan). The slit size of the energy filter was set to 20 eV. 13,800 micrographs were recorded at the 105,000× nominal magnification (corresponding to 0.873 Å per pixel) using 3 by 3 image shift, with the nominal defocus from −1.0 μm to −2.5 μm. Total 60 electrons were exposed per Å² during 4.2 seconds, fractionated into 60 frames.

### Image Processing

Warp (Tegunov and Cramer, 2019) was used for beam-induced motion correction and CTF estimation the total 13,800 movies. 12,024 Micrographs that had 5 Å or higher CTF-fit resolution were imported to cryoSPARC (Punjani et al., 2017) for further processing. Initial particle picking was done using template-based picking in cryoSPARC, followed by 2D classification. Resulting 38,807 particles from 2D averages with high-resolution features were used to train topaz (Bepler et al., 2019) neural network. This trained network was applied to the filtered 12,024 micrographs to extract initial 1,338,135 particle picks. 2D classification was followed to remove “junk” particles, resulting in 1,075,078 particles from the selected 2D averages, which were then re-extracted using RELION (Scheres, 2012) with Fourier-cropping (420 pixels to 128 pixels, corresponding to 2.86 Aper pixel). This particle stack was subjected to 3D classification in RELION, which yielded 237,671 particles of intact Cascade-TniQ complex. This particle stack was then re-extracted without Fourier cropping (0.873 Å per pixel), followed by non-uniform refinement and heterogeneous refinement in cryoSPARC. One resulting class (53%, 126,320 particles) from the heterogeneous refinement showed significantly stronger TniQ density, thus selected for the downstream analysis. We noticed that even after the two rounds of classifications, resulting particle stack incorporated considerable level of conformational heterogeneity.

Following the non-uniform refinement of the selected class (126,320 particles), 3D variability analysis (Punjani and Fleet, 2021) (3DVA) in cryoSPARC was used to analyze the conformational dynamics within the dataset. 3DVA visualization tool from cryoSPARC was used to cluster the input particle stack into three classes based on the identified eigenvectors and coordinates in the defined conformational space from the 3DVA. Each extreme of the space were identified as Cascade-TniQ complex with partial (28%, 34,800 particles) or full (20%, 26,306 particles) R-loop respectively, with 52% of particles in between showing disordered TniQ density. 34,800 particles that represent partial R-loop complex were then exported to RELION for Bayesian polishing (Zivanov et al., 2019), and CTF refinement (Zivanov et al., 2018) of the complex, which resulted in 4.0 Å resolution of partial R-loop complex. In order to maximize the number of particles in the class of full R-loop complex, the earlier stack of 126,320 particles was subjected to two independent heterogeneous refinements. Each refinement job was set up using two volumes of the extreme clusters from the 3DVA eigenvector 1 and eigenvector 2 respectively. Each refinement resulted in 57% or 58% of Cascade-TniQ with full R-loop respectively, which were then merged and deduplicated. This stack of 91,051 particles was exported to RELION. Signals outside of the PAM-distal region is subtracted using the mask that includes Cas6, TniQ dimer, Cas8 helix bundle, and PAM-distal DNA (FIG. 7B). Focused classification of the subtracted particles resulted in two major classes of PAM-distal DNA bound TniQ, but one class of high-resolution features (58%, 53,353 particles) was selected as the final particle stack for the Cascade-TniQ with full R-loop. CTF-refinement and Bayesian polishing of the particles resulted in 3.5 Å resolution of full R-loop complex. Focused refinement of the PAM-distal region resulted in 3.9 Å resolution. Final maps were sharpened using RELION postprocessing tool with automatically estimated B-factors. Local resolution estimation and filtering of the final reconstructions were done using cryoSPARC. For visualization, reconstructions from global and focused refinement were aligned and combined using UCSF Chimera (Pettersen et al., 2004) command ‘fitmap’ and ‘vop maximum’ respectively.

### Model Building and Validation

The atomic models for Cas8/5, Cas6, Cas7, and TniQ from a homologous Cascade-TniQ complex from V. cholerae (PDB: 6PIF) (Halpin-Healy et al., 2020) were used as templates for homology models generated using the I-TASSER server (Yang and Zhang, 2015). These homology models were docked in the cryo-EM density map using UCSF Chimera (Pettersen et al., 2004) and residue registers and backbone geometries were then corrected using Coot (Emsley et al., 2010). Extended loops that could not be modeled manually due to locally poorer EM density were rebuilt into the cryo-EM density maps using RosettaES (Frenz et al., 2017) and then manually refined in Coot in an iterative fashion. Refined protein models were subsequently relaxed into the EM density using Rosetta and were subjected to iterative rounds of relaxation in Rosetta and refinement in Coot to fix geometric and steric outliers identified by MolProbity (Williams et al., 2018) during model validation. crRNA and DNA strands were manually built into the cryo-EM density map using Coot with resolution sufficient to distinguish purines and pyrimidines and, thus, confirm the register of each strand. Nucleic acid models were refined into an EM map ‘zoned’ (using UCSF Chimera) to remove protein density and using phenix (Afonine et al., 2018; Echols et al., 2012) real_space_refine subject to base-pair and stacking restraints generated by inspection. Details of the validation stats are summarized in Table Si.

### Rosetta Simulation of PAM Specificity

PDB models for the DNA-bound I-F1 Cascade (PDB: 6NE0) (Rollins et al., 2019) and the full R-loop complex of the I-F3b system were used as inputs for specificity calculations. To generate the model of I-F3b A247Q S248N mutant, fixbb application of the Rosetta modeling suite was used beforehand (Dantas et al., 2003; Hu et al., 2007; Kuhlman et al., 2003; Leaver-Fay et al., 2005). This application was used to fit an amino acid rotamer onto the fixed backbone of an input structure at specific positions. In order to generate 16 models of each system with 16 possible PAM combinations, −1 and −2 position nucleotides were substituted using the Simple Mutate tool from coot (Emsley et al., 2010), which replaces the base identity from the original model without altering other geometries. Molecular modeling suite Rosetta was used to calculate the specificity of each system with each possible PAM sequence. RosettaScripts application (Fleishman et al., 2011) was used to both optimize binding and generate specificity values. For I-F1 Cascade and I-F3b wild-type, optimize binding, we allowed for rotamer packing of the residues at 247 and 248 position and backbone movement of this two-residue span and the four residues flanking either side of this region (total of 10 adjacent residues). Rotamer packing was specifically optimized to improve binding between the residue pair and the PAM base pairs using a DNA-based energy function (Ashworth and Baker, 2009; Ashworth et al., 2006; Ashworth et al., 2010; Thyme et al., 2009). Total energy scores for each structure-PAM model were calculated after rotamer/backbone optimization. This process was repeated to yield ten energy scores for each structure-PAM combination, which were then averaged to be used as a Boltzmann energy term. The specificity of each design model was then calculated as a Boltzmann occupancy that compared the target structure against a partition function consisting of all competing PAM combinations.

### Plasmid Construction

Plasmids for protein expression/purification, transposition and interference assays were constructed by standard methods including restriction/ligation, isothermal assembly, and golden gate cloning. F plasmid derivatives were made by recombineering as described in Petassi et al. 2020 The randomized 2-bp target plasmid for unbiased screening was constructed by amplification of plasmid backbone using primers with synthetic tails adding an ‘NN’-ffs target sequence and XhoI cut sites, digestion with XhoI and self-ligation.

### Mate-Out Transposition Assay

Mate-out assays were performed in strain MTP1043. Cells were made competent by growing in LB media to mid-log and washing/resuspending in ice cold CaCl₂) solution (Peters, 2007) and transformed with pMTP1293 (TnsABC), pMTP1261 (TniQ-Cascade) or mutant derivatives, and pMTP1379 (atypical crRNA targetingffs) or pMTP1382 (typical crRNA targetingffs) onto LB agar supplemented with 100 g/mL carbenicillin, 30 g/mL chloramphenicol, 8 g/mL tetracycline, and 0.2% w/v glucose. After 16 hours incubation at 37° C., several hundred transformants were washed up in M9 minimal media supplemented with 0.2% w/v maltose and diluted to a calculated OD=0.3 in M9 maltose supplemented with 100 g/mL carbenicillin, 30 g/mL chloramphenicol, 8 g/mL tetracycline, 0.2% w/v arabinose, and 100 M IPTG to induce transposition.

After 24 hours incubation with shaking at 30° C., a portion of induced cultures were washed once and resuspended in LB supplemented with 0.2% w/v glucose. After 1.5 hours incubation at 37° C. induced pools were mixed with prepared mid-log CW51 recipient strain at a ratio of 1:5 donor:recipient and incubated with gentle agitation for 90 minutes at 37° C. to allow mating. Cultures were then vortexed, placed on ice, serially diluted in LB 0.2% w/v glucose, and plated on LB supplemented with 20 g/mL nalidixic acid, 100 g/mL rifampicin, 100 g/mL spectinomycin, 50 g/mL X-gal, with or without 50 g/mL kanamycin to sample the entire transconjugant population or select for transposition respectively. Plates were incubated at 37° C. for 36 hours before colonies were counted.

P. aeruginosa CRISPR Interference Assay

Interference assays were performed in BL21-AL. BL21-AI was made competent by standard chemical methods (Peters, 2007) and transformed with pOPO322 (cas1_cas2/3), pCsy_complex, and pOPO374 (PA14 crRNA-ffs) onto LB agar supplemented with 100 g/mL carbenicillin, 100 g/mL spectinomycin, 30. g/mL chloramphenicol, and 0.2% w/v glucose. Overnight cultures grown in LB agar supplemented with 100 g/mL carbenicillin, 100 g/mL spectinomycin, 30 g/mL chloramphenicol were diluted 1:50 in LB supplemented with 100 g/mL carbenicillin, 100 g/mL spectinomycin, 30 g/mL chloramphenicol, 100 M IPTG and 1 mM arabinose. Cultures were grown to OD=0.4 before electrocompetent cells were prepared by standard methods (Peters, 2007) and transformed with 1 ng pOPO275 (CC-ffs target plasmid), alternate PAM derivative of pOPO275 or pOPO390 (non-target control). Cells were recovered in SOC at 37° C. for one hour before being serially diluted and plated on LB supplemented with 100 g/mL carbenicillin, 50 g/mL kanamycin, 30 g/mL chloramphenicol, and 100 g/mL spectinomycin. Plates were incubated at 37° C. for 16 hours before colonies were counted.

### Randomized PAM Transposition and Interference Assay

For Tn6900 unbiased PAM transposition screen, MTP997 (Escherichia coli BW27783 attTn7::miniTn7(miniTn6900(kanR))) transformed with pMTP1293 (TnsABC), pMTP1261 (TniQ-Cascade) or mutant derivatives, and pMTP1379 was made competent by standard chemical means (Peters, 2007) and transformed with pMTP1412 (NN-ffs target plasmid). >50,000 colonies were washed up, washed and resuspended to a calculated OD=0.3 in M9 maltose induction media (M9 maltose supplemented with 100 g/mL carbenicillin, g/mL chloramphenicol, 8 g/mL tetracycline, 10 g/mL gentamycin, 0.2% w/v arabinose, and 100 M IPTG) and induced for 24 hours @30 C before plasmids were purified with Omega E.Z.N.A. Plasmid DNA Mini Kit.

To remove self-targeting transposition into the guide-RNA expression vector contaminating transformations, plasmid pools were digested with EcoRI and SalI before transformation into DH5alpha onto LB supplemented with 10 g/mL gentamycin and 50 g/mL kanamycin to select for target plasmid with mini-transposon. >10,000 colonies were washed up, combined, plasmids purified and 2×151 bp read Illumina total DNA sequencing was done by MiGS (Microbial Genome Sequencing Center).

For I-F1 PA14 CRISPR-Cas unbiased PAM interference screen, BL21-AI transformed with pOPO322 (pACYCDuet-cas1_cas2_3), pCsy_complex, and pOPO374 (pCDFDuet-PA14-ffs) was grown overnight in LB agar supplemented with 100 g/mL carbenicillin, 100 g/mL spectinomycin, 30 g/mL chloramphenicol then diluted 1:50 in LB supplemented with 100 g/mL carbenicillin, 100 g/mL spectinomycin, 30 g/mL chloramphenicol, 100 μM IPTG and 1 mM arabinose. Cultures were grown to OD=0.4 before electrocompetent cells were prepared by standard methods and transformed with 1 ng pMTP1412 (pBBR-genR-NNffs). Cells were recovered in SOC at 37° C. for one hour before being plated on LB supplemented with 100 g/mL carbenicillin, 50 g/mL kanamycin, 30 g/mL chloramphenicol, and 100 g/mL spectinomycin. Plates were incubated at 37° C. for 16 hours before >10,000 colonies were washed up, plasmids purified and 2×151 bp read Illumina total DNA sequencing was done by MiGS.

Reads were processed by custom python code to extract and count reads containing each PAM position for the plasmid pool before and after transposition/interference assay. Data is plotted as heatmaps using matplotlib/seaborn (Hunter, 2007; Waskom, 2021) and PAM wheels using Krona (Leenay et al., 2016; Ondov et al., 2011)

TABLE S1
Cryo-EM data collection, refinement, and validation statistics.

| | Name | |
|---|---|---|
| | Cascade- | Cascade- |
| | TniQ with | TniQ with |
| | atypical crRNA, | atypical crRNA, |
| | full R-loop | partial R-loop |
| | PDB ID | |
| | 7U5D | 7U5E |
| EMDB ID | EMD-26348 | EMD-26349 |
| Data collection and Processing | | |
| Microscope | Titan Krios | Titan Krios |
| Voltage (keV) | 300 | 300 |
| Camera | K3 | K3 |
| Magnification | 105,000 | 105,000 |
| Pixel size at detector (Å/pixel) | 0.873 | 0.873 |
| Total electron exposure (e⁻/Å²) | 60 | 60 |
| Exposure rate (e⁻/pixel/sec) | 10.89 | 10.89 |
| Number of frames collected | 60 | 60 |
| Defocus range (μm) | −1.0-−2.5 | −1.0-−2.5 |
| Automation software | SerialEM | SerialEM |
| Energy filter slit width | 20 keV | 20 keV |
| Micrographs collected (no.) | 13,800 | 13,800 |
| Micrographs used (no.) | 12,024 | 12,024 |
| Total extracted particles (no.) | 1,338,135 | 1,338,135 |
| For each reconstruction: | | |
| Refined particles (no.) | 126,320 | 126,320 |
| Final particles (no.) | 53,353 | 34,800 |
| Symmetry | C1 | C1 |
| Resolution (global, Å) | | |
| FSC 0.5 (unmasked/masked) | 7.48/4.17 | 8.94/4.95 |
| FSC 0.143 (unmasked/masked) | 4.26/3.52 | 4.95/4.03 |
| Resolution range (local, Å) | 3.3-7.0 | 3.4-7.0 |
| Resolution range due to | 3.30-3.86 | 3.78-4.64 |
| anisotropy (Å) | | |
| Map sharpening B factor (Å²) | −70 | −81 |
| Map sharpening methods | RELION | RELION |
| Model composition | | |
| Protein residues | 3,603 | 3,474 |
| Ligands | 0 | 0 |
| RNA/DNA | 141 | 100 |
| Model Refinement | | |
| Refinement package | Coot/Rosetta/ | Coot/Rosetta/ |
| | Phenix | Phenix |
| real or reciprocal space | Real | Real |
| resolution cutoff (Å) | 3.5 | 4.0 |
| Model-Map scores | | |
| CC (box) | 0.81 | 0.77 |
| Average FSC (0.5 cutoff, Å) | 3.71 | 4.30 |
| R.m.s deviations from ideal | | |
| values | | |
| Bond length (Å) | 0.012 | 0.012 |
| Bond angles (°) | 1.613 | 1.616 |
| Validation | | |
| MolProbity score | 1.74 | 1.78 |
| CaBLAM outliers (%) | 2.15 | 2.26 |
| Clashscore | 10.15 | 11.26 |
| Poor rotamers (%) | 0.20 | 0.14 |
| C-beta outliers (%) | 0.03 | 0.03 |
| EMRinger score | 2.90 | 2.06 |
| Ramachandran plot | | |
| Favored (%) | 96.66 | 96.66 |
| Outliers (%) | 0.17 | 0.12 |

The following references relate to Example 6. This reference listing is not an indication that any reference is material to patentability.

### REFERENCES

Afonine, P. V., Klaholz, B. P., Moriarty, N. W., Poon, B. K., Sobolev, O. V., Terwilliger, T. C., Adams, P. D., and Urzhumtsev, A. (2018). New tools for the analysis and validation of cryo-EM maps and atomic models. Acta Crystallogr D Struct Biol 74, 814-840.
Ashworth, J., and Baker, D. (2009). Assessment of the optimization of affinity and specificity at protein-DNA interfaces. Nucleic Acids Res 37, e73.
Ashworth, J., Havranek, J. J., Duarte, C. M., Sussman, D., Monnat, R. J., Jr., Stoddard, B. L., and Baker, D. (2006). Computational redesign of endonuclease DNA binding and cleavage specificity. Nature 441, 656-659.
Ashworth, J., Taylor, G. K., Havranek, J. J., Quadri, S. A., Stoddard, B. L., and Baker, D. (2010). Computational reprogramming of homing endonuclease specificity at multiple adjacent base pairs. Nucleic Acids Res 38, 5601-5608.
Bepler, T., Morin, A., Rapp, M., Brasch, J., Shapiro, L., Noble, A. J., and Berger, B. (2019). Positive-unlabeled convolutional neural networks for particle picking in cryo-electron micrographs. Nat Methods 16, 1153-1160.
Dantas, G., Kuhlman, B., Callender, D., Wong, M., and Baker, D. (2003). A large scale test of computational protein design: folding and stability of nine completely redesigned globular proteins. J Mol Biol 332, 449-460.
Echols, N., Grosse-Kunstleve, R. W., Afonine, P. V., Bunkoczi, G., Chen, V. B., Headd, J. J., McCoy, A. J., Moriarty, N. W., Read, R. J., Richardson, D. C., et al. (2012). Graphical tools for macromolecular crystallography in PHENIX. J Appl Crystallogr 45, 581-586.
Emsley, P., Lohkamp, B., Scott, W. G., and Cowtan, K. (2010). Features and development of Coot. Acta Crystallogr D Biol Crystallogr 66, 486-501.
Fleishman, S. J., Leaver-Fay, A., Corn, J. E., Strauch, E. M., Khare, S. D., Koga, N., Ashworth, J., Murphy, P., Richter, F., Lemmon, G., et al. (2011). RosettaScripts: a scripting language interface to the Rosetta macromolecular modeling suite. PLoS One 6, e20161.
Frenz, B., Walls, A. C., Egelman, E. H., Veesler, D., and DiMaio, F. (2017). RosettaES: a sampling strategy enabling automated interpretation of difficult cryo-EM maps. Nat Methods 14, 797-800.
Halpin-Healy, T. S., Klompe, S. E., Sternberg, S. H., and Fernandez, I. S. (2020). Structural basis of DNA targeting by a transposon-encoded CRISPR-Cas system. Nature 577, 271-274.
Hu, X., Wang, H., Ke, H., and Kuhlman, B. (2007). High-resolution design of a protein loop. Proc Natl Acad Sci USA 104, 17668-17673.
Hunter, J. D. (2007). Matplotlib: A 2D graphics environment. Computing in science & engineering 9, 90-95.
Kuhlman, B., Dantas, G., Ireton, G. C., Varani, G., Stoddard, B. L., and Baker, D. (2003). Design of a novel globular protein fold with atomic-level accuracy. Science 302, 1364-1368.
Leaver-Fay, A., Kuhlman, B., and Snoeyink, J. (2005). An adaptive dynamic programming algorithm for the side chain placement problem. Pac Symp Biocomput, 16-27.
Leenay, R. T., Maksimchuk, K. R., Slotkowski, R. A., Agrawal, R. N., Gomaa, A. A., Briner, A. E., Barrangou, R., and Beisel, C. L. (2016). Identifying and visualizing functional PAM diversity across CRISPR-Cas systems. Molecular cell 62, 137-147.
Ondov, B. D., Bergman, N. H., and Phillippy, A. M. (2011). Interactive metagenomic visualization in a Web browser. BMC bioinformatics 12, 1-10.
Peters, J. E. (2007). Gene Transfer in Gram-Negative Bacteria. Methods for general and molecular microbiology, 735-755.
Pettersen, E. F., Goddard, T. D., Huang, C. C., Couch, G. S., Greenblatt, D. M., Meng, E. C., and Ferrin, T. E. (2004). UCSF Chimera—a visualization system for exploratory research and analysis. J Comput Chem 25, 1605-1612.
Punjani, A., and Fleet, D. J. (2021). 3D variability analysis: Resolving continuous flexibility and discrete heterogeneity from single particle cryo-EM. J Struct Biol 213, 107702.
Punjani, A., Rubinstein, J. L., Fleet, D. J., and Brubaker, M. A. (2017). cryoSPARC: algorithms for rapid unsupervised cryo-EM structure determination. Nat Methods 14, 290-296.
Rollins, M. F., Chowdhury, S., Carter, J., Golden, S. M., Miettinen, H. M., Santiago-Frangos, A., Faith, D., Lawrence, C. M., Lander, G. C., and Wiedenheft, B. (2019). Structure Reveals a Mechanism of CRISPR-RNA-Guided Nuclease Recruitment and Anti-CRISPR Viral Mimicry. Mol Cell 74, 132-142 e135.
Scheres, S. H. (2012). RELION: implementation of a Bayesian approach to cryo-EM structure determination. J Struct Biol 180, 519-530.
Tegunov, D., and Cramer, P. (2019). Real-time cryo-electron microscopy data preprocessing with Warp. Nat Methods 16, 1146-1152.
Thyme, S. B., Jarjour, J., Takeuchi, R., Havranek, J. J., Ashworth, J., Scharenberg, A. M., Stoddard, B. L., and Baker, D. (2009). Exploitation of binding energy for catalysis and design. Nature 461, 1300-1304.
Waskom, M. L. (2021). Seaborn: statistical data visualization. Journal of Open Source Software 6, 3021.
Williams, C. J., Headd, J. J., Moriarty, N. W., Prisant, M. G., Videau, L. L., Deis, L. N., Verma, V., Keedy, D. A., Hintze, B. J., Chen, V. B., et al. (2018). MolProbity: More and better reference data for improved all-atom structure validation. Protein Sci 27, 293-315.
Yang, J., and Zhang, Y. (2015). I-TASSER server: new development for protein structure and function predictions. Nucleic Acids Res 43, W174-181.
Zivanov, J., Nakane, T., Forsberg, B. O., Kimanius, D., Hagen, W. J., Lindahl, E., and Scheres, S. H. (2018). New tools for automated high-resolution cryo-EM structure determination in RELION-3. Elife 7.
Zivanov, J., Nakane, T., and Scheres, S. H. W. (2019). A Bayesian approach to beam-induced motion correction in cryo-EM single-particle analysis. IUCrJ 6, 5-17.

1. One or more modified proteins for use in a CRISPR system to modify a DNA substrate, wherein the one or more proteins are selected from:
i) a Cas6 protein comprising an amino acid change relative to a reference amino acid sequence at position 113 or 153, and wherein optionally the change is F113A or F153A;
ii) a TniQ protein comprising one or more amino acid changes relative to a reference amino acid sequence at position 384, 387, 283, and 330, and wherein optionally the one or more changes are selected from H384A, H387A, N283A, and R330A;
iii) a Cas8/5 protein comprising one or more amino acid changes relative to a reference amino acid sequence at position 247 or 248, and wherein optionally the one or more changes are selected from A247T, A247Q, S248A, and S248N.

2. The modified protein of claim 1, wherein the modified protein comprises a combination of two of the amino acid changes.

3. The modified protein of claim 2, wherein the modified protein is a modified TniQ protein that comprises a combination of amino acid changes at positions 384 and 387.

4. The modified protein of claim 2, wherein the modified protein is a modified Cas8/5 protein that comprises a combination of amino acid changes at positions 247 and 248.

5. The modified protein of claim 1, wherein the reference amino acid sequence for at least one of the Cas6, the TniQ, and the Cas8/5 protein is from Cas6, TniQ, and Cas8/5 amino acid sequences encoded by Tn6900 element from Aeromonas salmonicida S44.

6. A polynucleotide encoding a modified protein or combination of modified proteins as in claim 1.

7. A system for use in modifying a DNA substrate, the system comprising one or more modified proteins of claim 1, or one or more polynucleotides encoding the one or more modified proteins, the system further comprising Cas6, Cas7, TnsA, TnsB, and TnsC proteins, or polynucleotides encoding said Cas6, Cas7, TnsA, TnsB, and TnsC proteins.

8. The system of claim 7, further comprising a guide RNA that targets a selected location in a chromosome or plasmid.

9. The system of claim 8, wherein said system exhibits one or more improved DNA modification properties relative to DNA modification using one or more unmodified versions of the Cas6, the TniQ, or the Cas8/5 protein, or a combination thereof.

10. The system of claim 9, wherein the one or more improved DNA modification properties are selected from: improved transposition using a typical guide RNA, improved transposition using an atypical guide RNA, increased protospacer adjacent motif (PAM) specificity, or a combination thereof.

11. The system of claim 10, further comprising a double stranded DNA template for introduction into a selected location of a chromosome or plasmid.

12. A method comprising introducing a system of claim 11 into cells such that the DNA template is introduced into the selected location of the cells.

13. The method of claim 12, wherein introduction of the DNA template exhibits one or more improved properties relative to introduction of the same DNA template using one or more unmodified versions of the Cas6, the TniQ, or the Cas8/5 protein.

14. The method of claim 13, wherein the one or more improved properties are selected from improved frequency of introduction of the doubled stranded DNA template using a typical guide RNA, improved frequency of introduction of the doubled stranded DNA template using an atypical guide RNA, increased PAM specificity for introduction of the doubled stranded DNA template, or a combination thereof.
